(19) United States
(12) Patent Application Publication
(10) Pub. No.: US 2015/0344952 A1
(43) Pub. Date: Dec. 3, 2015

(54) DNA MARKERS FOR BEEF TENDERNESS IN CATTLE

(71) Applicant: The Texas A&M University System, College Station, TX (US)

(72) Inventors: PENNY K. RIGGS, Bryan, TX (US); Robert K. Vaughn, College Station, TX (US)

(21) Appl. No.: 14/724,690

(22) Filed: May 28, 2015

Related U.S. Application Data
(60) Provisional application No. 62/006,737, filed on Jun. 2, 2014.

(51) Int. Cl. C12Q 1/68

(52) U.S. Cl. CPC C12Q 1/6881; C12Q 2600/156; C12Q 2600/124

(57) ABSTRACT

The present invention provides genetic polymorphisms and methods for identifying said genetic polymorphisms associated with desired beef tenderness in cattle, as well as kits for identifying said polymorphisms in beef cattle. The invention also provides methods of predicting the tenderness of beef in a head of cattle before slaughter based on the presence of a hapblock conferring tenderness. In other embodiments, the invention provides methods of determining a breeding value for a head of cattle involving detection of such polymorphisms.

### CROSS REFERENCE TO RELATED APPLICATIONS

This application claims priority to U.S. Provisional Application No. 62/006,737, filed Jun. 2, 2014, which is herein incorporated by reference in its entirety.

### FIELD OF THE INVENTION

The present invention relates generally to the field of mammalian genetics. More particularly, the invention concerns genetic markers for the selection of cattle having a genetic predisposition for progeny with superior beef tenderness traits.

### INCORPORATION OF SEQUENCE LISTING

The sequence listing that is contained in the file named “TAMC030US_ST25,” which is 13 kilobytes as measured in Microsoft Windows operating system and was created on May 26, 2015, is filed electronically herewith and incorporated herein by reference.

### BACKGROUND OF THE INVENTION

Genetic improvement for meat quality or the efficiency of production is needed for the improvement of beef cattle traits. In particular, tenderness in beef is the main factor affecting consumer palatability ratings. In consumer sensory panel studies, tenderness accounts for more than 50% of the total value placed on meat by consumers, who have expressed a willingness to pay a premium price for reliably labeled tender beef. This preference makes tenderness an issue of concern to producers and consumers alike. However, breeding cattle with improved tenderness has been difficult to date in view of problems associated with phenotypically assessing tenderness while animals are still alive and capable of being bred, as well as poorly understood inheritance of the tenderness trait. There is therefore a great need for development of indirect measures for selection of beneficial traits in beef cattle, such as DNA diagnostics.

### SUMMARY OF THE INVENTION

In one aspect, the invention provides a method of selecting a head of beef cattle with a genetic predisposition for increased meat tenderness comprising selecting said head of beef cattle based on the presence in the genome of at least one genetic haplotype conferring said increased meat tenderness selected from the group consisting of (a) a haplotype located in a region on bovine chromosome 2 defined by positions −24230649 and −24118992; (b) a haplotype located in a region on bovine chromosome 2 defined by positions 103881402 and 103950562; and (c) a haplotype located in a region on bovine chromosome 2 defined by positions 36242459 and 36362623. In certain embodiments, said haplotype of (a) comprises the ITGA6 gene, or said haplotype of (b) comprises the FN1 gene, or said haplotype of (c) comprises the ITGB6 gene. In another embodiment, such a method comprises selecting said head of beef cattle based on the presence in the genome of at least two of said haplotypes. In other embodiments, said haplotype of (a) is inherited from the maternal or the paternal parent, or is inherited from the genome of a Nellore head of cattle. In other embodiments, said haplotype of (b) is inherited from the paternal parent, or is inherited from the genome of an Angus head of cattle. In still further embodiments, said haplotype of (c) is inherited from the maternal parent or the paternal parent, or is inherited from the genome of a Nellore head of cattle. In another embodiment, such a method further comprises sequencing said haplotype. In other embodiments, the method further comprises detecting at least one SNP within or genetically linked to said at least one genetic haplotype, wherein said SNP is set forth in Tables 12-14, or further comprises detecting all SNPs within or genetically linked to said haplotype.

In still other embodiments, the method further comprises genotyping at least one parent of said head of beef cattle for the presence of said at least one genetic haplotype, or genotyping both parents of said head of beef cattle. In other embodiments, said head of beef cattle is a Bos indicus or a Bos taurus head of beef cattle, or is a hybrid of a Bos indicus species and a Bos taurus species. In another embodiment, said Bos indicus species further comprises a Nellore head of cattle, or said Bos taurus species further comprises an Angus head of cattle. In another embodiment, the method further comprises genotyping a population of beef cattle for the presence of said at least one genetic haplotype. In other embodiments, the method further comprises breeding the selected head of beef cattle comprising said at least one genetic haplotype with a second head of beef cattle to obtain a progeny head of beef cattle with desired meat tenderness relative to a head of beef cattle of the same breed lacking said at least one genetic haplotype, or breeding the progeny head of beef cattle with desired meat tenderness to a second head of beef cattle to produce a progeny of a further generation comprising said desired meat tenderness. In other embodiments, the presence of said at least one genetic haplotype conferring said increased meat tenderness is detected by assaying of genetic material from the head of beef cattle, or said assaying is carried out by PCR.

In another aspect, the invention provides a method of predicting the tenderness of meat in a head of beef cattle after electrical stimulation comprising genotyping the head of beef cattle for the presence in the genome of at least one genetic haplotype conferring said increased meat tenderness selected from the group consisting of (a) a haplotype located in a region on bovine chromosome 2 defined by positions −24230649 and −24118992; (b) a haplotype located in a region on bovine chromosome 2 defined by positions 103881402 and 103950562; and (c) a haplotype located in a region on bovine chromosome 2 defined by positions 36242459 and 36362623. In some embodiments, said head of beef cattle is a Bos indicus or a Bos taurus head of beef cattle, or said head of beef cattle is a hybrid between a Bos indicus species and a Bos taurus species, or said Bos indicus species further comprises a Nellore head of cattle, or said Bos taurus species further comprises further comprises an Angus head of cattle.

In another aspect, the invention provides a method of determining the breeding value of a head of cattle comprising: (a) genotyping the head of beef cattle to determine the presence in the genome of at least one genetic haplotype conferring said increased meat tenderness selected from the group consisting of (i) a haplotype located in a region on bovine chromosome 2 defined by positions −24230649 and −24118992; (ii) a haplotype located in a region on bovine chromosome 2 defined by positions 103881402 and 103950562; and (iii) a haplotype located in a region on bovine chromosome 2 defined by positions 36242459 and 36362623; (b) determining an estimated breeding value for the individual; and (c) selecting at least a first individual with a desired estimated breeding value for breeding. In one embodiment, the method further comprises (d) breeding said individual to a second individual to obtain progeny.

In another aspect, the invention provides a kit for identifying a head of beef cattle with increased meat tenderness following postmortem electrical stimulation, the kit comprising: (a) at least one primer that amplifies a SNP within or genetically linked to at least one haplotype conferring said increased meat tenderness selected from the group consisting of (i) a haplotype located in a region on bovine chromosome 2 defined by positions −24230649 and −24118992; (ii) a haplotype located in a region on bovine chromosome 2 defined by positions 103881402 and 103950562; and (ii) a haplotype located in a region on bovine chromosome 2 defined by positions 36242459 and 36362623; wherein said SNP is set forth in Tables 12-14; and (b) a reaction reagent.

### BRIEF DESCRIPTION OF THE DRAWINGS

FIG. 1—Shows a graph demonstrating the Warner-Bratzler shear force (WBSF) for a hapblock encompassing the ITGA6 gene. A 1-Mb region encompassing ITGA6 was examined and cattle were genotyped for alleles of the hapblock. Left bar: increased shear force for cattle having 2 Angus hapblock alleles. Middle bar: decreased shear force for a heterozygote having one Angus hapblock allele, and one Nellore hapblock. Right bar: lowest shear force was obtained for cattle having 2 Nellore hapblock alleles. Each Angus hapblock accounted for approximately 7% increased meat toughness.

FIG. 2—Shows a graph demonstrating the Warner-Bratzler shear force (WBSF) for a hapblock encompassing the FN1 gene. A 1-Mb region encompassing FN1 was examined and cattle were genotyped for alleles of the hapblock. Left bar: decreased shear force for cattle having an Angus hapblock allele. Right bar: higher shear force for cattle having a Nellore hapblock allele.

FIG. 3—Shows a graph demonstrating the Warner-Bratzler shear force (WBSF) for a hapblock encompassing the ATGB6 gene. A 1-Mb region encompassing ATGB6 was examined and cattle were genotyped for alleles of the hapblock. Left bar: increased shear force for cattle having 2 Angus hapblock alleles. Middle bar: decreased shear force for a heterozygote having one Angus hapblock allele, and one Nellore hapblock. Right bar: lowest shear force was obtained for cattle having 2 Nellore hapblock alleles.

FIG. 4—Shows western blot analyses of samples grouped by Warner-Bratzler shear force (WBSF) electrical stimulation (ES) residuals. A 5% polyacrylamide stacking gel and 10% resolving gel were used.

### BRIEF DESCRIPTION OF THE SEQUENCES

SEQ ID NOs:1-78—Sequences of primers used for qRT-PCR.

### DETAILED DESCRIPTION OF THE INVENTION

The invention provides, in one aspect, methods and compositions for the improvement of beef cattle with respect to tenderness of the beef obtained from the cattle after slaughter. The invention provides for the first time genetic haplotypes harboring detectable single nucleotide polymorphisms (SNPs) and that confer increased tenderness in beef cattle. Thus, in one embodiment, the invention provides a method of selecting a head of beef cattle with increased meat tenderness comprising selecting a head of beef cattle based on the presence in the genome of at least one genetic marker indicating the presence of a genetic haplotype conferring increased meat tenderness.

As described in the Examples below, genetic loci (haplotype blocks or hapblocks) were identified that harbor SNPs that can serve as a predictive test for tenderness by enabling a cattle breeder to identify a head of cattle that will produce beef with desirable tenderness. Phenotypic assays for tenderness previously were generally carried out on a carcass after slaughter, preventing implementation of that information into a breeding program for development of improved cattle. In contrast, the applicants found that the hapblocks described herein are heritable and thus can be successfully introduced into any desired cattle genetic background or breed. Previous genetic tests for tenderness in beef were based on components of a proteolytic pathway believed to be involved in postmortem tenderization. These genes were evaluated in the present study and found to be of value for detecting predisposition to beef tenderness as described herein. The present invention thus represents a significant advantage by identifying new loci that can be tracked with genetic markers in beef cattle to yield a head of beef cattle with increased beef tenderness relative to cattle lacking the relevant haplotype.

Bos indicus, a species of beef cattle, indigenous to the southern U.S., in particular Texas, has traditionally been considered less desirable to consumers, as the meat has the perception of reduced meat quality, particularly tenderness. British cattle breeds, including breeds from Bos taurus, such as Angus are generally preferred by consumers based on the tenderness of the beef, although these breeds lack the productivity of native breeds. Crossbred cattle such as those produced with indicine beef breeds (i.e., cattle from the species B. indicus) can therefore provide a benefit by enabling breeders to produce cattle with desirable tenderness in the beef, while maintaining the beneficial productivity of native cattle breeds.

Applying an electrical current to a beef carcass postmortem is one commonly employed commercial method for improving end-product tenderness. Electrical stimulation (ES) has been shown to reduce variation in tenderness between carcasses. High voltage ES has been shown to overcome some of the effects of breed on tenderness, thus enabling B. indicus-influenced carcasses to reach a level of tenderness more consistent with B. taurus carcasses under the same postmortem conditions. Thus, in one embodiment, the methods of the invention provide for pre-slaughter identification of a head of beef cattle that will produce more tender beef following postmortem ES. Such a method may serve as a genetic test for predicting the tenderness of beef from a head of cattle prior to slaughter based on the identification in the genome of the head of cattle of a haplotype or hapblock as described herein. Haplotypes such as those described herein, when present in a head of beef cattle, confer increased tenderness in the meat after slaughter when the meat is treated with ES. In one embodiment, a head of beef cattle in accordance with the invention may be subjected to postmortem ES. In another embodiment, such a head of cattle may be processed after slaughter without postmortem ES.

Such a haplotype may be selected from the group consisting of (a) a haplotype located in a region on bovine chromosome 2 defined by genome positions −24230649 and −24118992; (b) a haplotype located in a region on bovine chromosome 2 defined by positions 103881402 and 103950562; and (c) a haplotype located in a region on bovine chromosome 2 defined by positions 36242459 and 36362623, including any combinations thereof. In one embodiment, a head of cattle may comprise a single haplotype as set forth herein conferring increased meat tenderness. In another embodiment, a head of cattle may comprise more than one haplotype conferring increased meat tenderness. Also provided are methods of determining a breeding value for a head of beef cattle comprising (a) genotyping the head of beef cattle to determine the presence in the genome of at least one genetic haplotype conferring said increased meat tenderness as set forth herein; (b) determining an estimated breeding value for the individual; and (c) selecting at least a first individual with a desired estimated breeding value for breeding.

In accordance with the invention, a haplotype set forth herein conferring increased tenderness in beef cattle may be inherited from either the maternal parent or the paternal parent of a Bos indicus breed of cattle. In certain embodiments, inheritance of a haplotype described herein from a particular parent may confer increased meat tenderness relative to inheritance of the haplotype from the other parent. For example, in one embodiment, inheritance of a haplotype located in a region on bovine chromosome 2 defined by positions −24230649 and −24118992 may confer increased meat tenderness when inherited from the maternal parent or the paternal parent. In another embodiment, inheritance of a haplotype located in a region on bovine chromosome 2 defined by positions 103881402 and 103950562 may confer increased meat tenderness when inherited from the paternal parent. In another embodiment, inheritance of a haplotype located in a region on bovine chromosome 2 defined by positions 36242459 and 36362623 may confer increased meat tenderness when inherited from the maternal parent or the paternal parent. Inheritance from either parent, however, may be used in accordance with the invention to confer an improved phenotype.

In other embodiments, inheritance of a haplotype in accordance with the invention from a specific lineage or genome may confer increased meat tenderness relative to inheritance of the haplotype from another lineage or genome. For example, in one embodiment, inheritance of a haplotype located in a region on bovine chromosome 2 defined by positions −24230649 and −24118992 may confer increased meat tenderness when the haplotype originates from a B. indicus lineage or genome. In a specific embodiment, such a haplotype may be inherited from a Nellore breed of cattle. In another embodiment, inheritance of a haplotype located in a region on bovine chromosome 2 defined by positions 103881402 and 103950562 may confer increased meat tenderness when the haplotype originates from a B. taurus lineage or genome. In a specific embodiment, such a haplotype may be inherited from an Angus breed of cattle. In another embodiment, inheritance of a haplotype located in a region on bovine chromosome 2 defined by positions 36242459 and 36362623 may confer increased meat tenderness when the haplotype originates from a B. indicus lineage or genome. In a specific embodiment, such a haplotype may be inherited from a Nellore breed of cattle. Additional haplotypes conferring increased meat tenderness in accordance with the invention are also within the scope of the invention.

In further embodiments, a haplotype as described herein may comprise or be genetically linked to certain genes or markers useful in accordance with the present invention. For example, in an embodiment, a haplotype located in a region on bovine chromosome 2 defined by positions −24230649 and −24118992 conferring increased beef tenderness may comprise or be genetically linked to an integrin gene, such as a gene encoding integrin alpha-6 (ITGA6). In another embodiment, a haplotype located in a region on bovine chromosome 2 defined by positions 103881402 and 103950562 conferring increased beef tenderness may comprise or be genetically linked to a fibronectin gene, such as a gene encoding fibronectin 1 (FN1). In another embodiment, a haplotype located in a region on bovine chromosome 2 defined by positions 36242459 and 36362623 conferring increased beef tenderness may comprise or be genetically linked to an integrin gene, such as a gene encoding integrin beta-6 (ITGB6).

In certain other embodiments, the invention provides a genetic locus or haplotype from a Bos indicus or Bos taurus beef cattle breed that confers desirable or increased beef tenderness. This haplotype can be introduced into cattle of different cattle breeds or species to produce cattle that comprise the trait of desired or increased tenderness in the beef when the animal is slaughtered. The locus of interest can be bred into in any species or breed of cattle as set forth herein. One of skill in the art will understand that other breeds of beef cattle may be useful in accordance with the invention.

In accordance with the invention, a haplotype of interest in the present invention may comprise a polymorphism within or genetically linked to a haplotype set forth herein. For example, a polymorphism in accordance with the invention may comprise a polymorphism set forth herein as being located in or genetically linked to a region on bovine chromosome 2 defined by positions −24230649 and −24118992, or located in or genetically linked to a region on bovine chromosome 2 defined by positions 103881402 and 103950562, or located in or genetically linked to a region on bovine chromosome 2 defined by positions 36242459 and 36362623. For example, a polymorphism of the invention may comprise a SNP as set forth in Tables 12-14. In a specific embodiment, a haplotype or hapblock as described herein may comprise a plurality of polymorphisms or SNPs. Such SNPs may be inherited together to produce increased or desired beef tenderness. For example, a haplotype or hapblock may comprise a single SNP, or may comprise 2, 3, 4, 5, 6, 7, 8, 9, 10, 11, 12, 13, 14, 15, 16, 17, 18, 19, 20, or more SNPs that may be inherited together to produce increased or desired beef tenderness.

### I. Genetic Assays and Selections

The techniques of the invention are significant in that they allow improvement of beef cattle for a trait that is important to both consumers and producers of beef without the need for costly or unreliable phenotypic assays and manual breeding selections. With increasing costs associated with animal breeding and artificial insemination, each head of cattle produced represents a substantial investment of time and money. Traditional methods of breeding cattle can lack accuracy due to environmental variance or scoring error. Further, complex gene action and interactions among genes can complicate breeding. Phenotypic selection often does not efficiently take into account such genetic variability. Selection based on DNA tests is therefore significant in that it allows selection of beef cattle for meat with increased tenderness without the cost and lack of reliability of conventional assays or selections.

The use of genetic assays as described herein to identify a haplotype or polymorphism associated with increased meat tenderness will find use in breeding or selecting of beef cattle produced for slaughter, e.g., for production of meat products. Thus, one embodiment of the invention comprises a breeding program directed at enhancement of desirable characteristics in beef cattle breeds adapted for meat production, as opposed to cattle specifically suited or used for production of dairy products. Such techniques have to date been largely lacking for beef cattle.

Genetic assay-assisted selections for animal breeding are important in that they allow selections to be made without the need for raising and phenotypic testing of progeny. In particular, such tests allow selections to occur among related individuals that do not necessarily exhibit the trait in question and that can be used in introgression strategies to select both for the trait to be introgressed and against undesirable background traits. However, it is has been difficult to identify genetic assays for loci yielding highly heritable traits of large effect, particularly as many such traits may not be segregating and already be fixed with near optimal alleles in commercial lines. The invention overcomes this difficulty by providing such assays for alleles of hapblocks that are segregating in beef cattle populations.

In accordance with the invention, any assay which sorts and identifies animals based upon differences in hapblock alleles or SNPs within or genetically linked thereto may be used and is specifically included within the scope of this invention. One of skill in the art will recognize that, having identified a hapblock or associated polymorphism for a particular associated trait, there are an essentially infinite number of ways to genotype animals for this hapblock or polymorphism. These tests may be made at the nucleic acid and/or protein level. The design of such alternative tests merely represents a variation of the techniques provided herein and is thus within the scope of this invention as fully described herein. Illustrative procedures are described herein below, but one of skill in the art will recognize that other techniques or methods may also be used in accordance with the invention.

Samples obtained from an individual can be analyzed for the presence of hapblocks or SNPs associated with meat tenderness using any suitable method, including microarray. In addition, several methods and different genotyping platforms that are used for SNP genotyping are known in the art (e.g., TaqMan, Pyrosequencing, RFLP, Direct Sequencing, etc.). Microarrays or chips can contain thousands up to a million or a little over one million SNPs, which are also used for genotyping.

Alternatively, or in addition to, analyzing a sample for the presence of hapblocks or SNPs associated with meat tenderness, a sample may be analyzed for the presence of a gene in which a particular SNP resides, or a gene product encoded by a gene in which a particular SNP resides. Gene products as used herein refer to any molecule produced as a result of gene expression, and may include proteins, mRNAs, tRNAs, microRNAs, or the like. The at least one gene may include but is not limited to ITGA6, FN1, or ITGB6. In some embodiments, the at least one gene may comprise more than one or all three of these genes, or any number of genes identified as useful in accordance with the invention. Methods of analyzing a sample for the presence of a haplotype and/or SNP and/or gene and/or gene product are well known in the art. As described herein, such methods may include PCR for verifying the presence of a particular haplotype and/or gene and/or SNP, direct sequencing, or real-time PCR for detecting the expression of a gene in different cells or tissues.

Non-limiting examples of methods for identifying the presence or absence of a hapblock or polymorphism include single-strand conformation polymorphism (SSCP) analysis, RFLP analysis, heteroduplex analysis, denaturing gradient gel electrophoresis, temperature gradient electrophoresis, ligase chain reaction, and direct sequencing of the gene. Techniques employing PCR detection are advantageous in that detection is more rapid, less labor intensive and requires smaller sample sizes. The techniques and primers that may be used in this regard are well known in the art. A PCR-amplified portion of, for example, the ITGA6 gene, the FN1 gene, or the ITGB6 gene, or a surrounding region may be screened for a polymorphism, for example, with direct sequencing of the amplified region, by microarray, by detection of restriction fragment length polymorphisms produced by contacting the amplified fragment with a restriction endonuclease having a cut site altered by the polymorphism, by allele-specific PCR in which the alleles are individually amplified by specific oligonucleotide primers, by SSCP analysis of the amplified region, or by other methods known in the art. These techniques may also be carried out directly on genomic nucleic acids without the need for PCR amplification, although in some applications this may require more labor. In accordance with the invention, a region surrounding a gene of interest, such as ITGA6, FN1, or ITGB6, which may be useful for identification of SNPs linked to beef tenderness may be found within a distance of approximately 1 Mb of the gene of interest. For example, SNPs may be identified within about 1 kb, 2 kb, 3 kb, 4 kb, 5 kb, 10 kb, 15 kb, 20 kb, 25 kb, 50 kb, 75 kb, 100 kb, 200 kb, 250 kb, 500 kb, 750 kb, or 1 Mb or more from a gene of interest.

Once an assay format has been chosen, selections may be unambiguously made based on genotypes assayed at any time after a nucleic acid or protein sample can be collected from an individual, such as an adult, or a young animal, or even earlier in the case of testing of embryos in vitro, or testing of fetal offspring. Any source of genetic material (including, for example, DNA and RNA) or a product encoded or produced thereby may be analyzed for scoring of genotype. In one embodiment of the invention, nucleic acids are screened that have been isolated from the hair roots, blood, muscle tissue, or semen of the bovine analyzed. Peripheral white blood cells may be conveniently used as a source of genetic material, and the genetic material is DNA. A sufficient amount of cells are obtained to provide a sufficient amount of DNA for analysis, although only a minimal sample size will be needed where scoring is by amplification of nucleic acids. DNA can be isolated from blood cells or other cell or tissue types by standard nucleic acid isolation techniques known to those skilled in the art.

In genetic assay-assisted breeding, eggs may be collected from selected females and in vitro fertilized using semen from selected males and implanted into the selected female or into another female for gestation and birth. Assays may be advantageously used with both male and female cattle. Using in vitro fertilization, genetic assays may be conducted on developing embryos at the 4-8 cell stage, for example, using PCR, and selections made accordingly. Embryos can thus be selected that are homozygous or heterozygous for the desired haplotype or marker prior to embryo transfer.

Alternatively, genetic assay-assisted breeding may comprise artificial insemination methods. In particular, semen from a selected male may be artificially introduced to the reproductive tract of a selected female to result in in vivo fertilization. Use of genotype-assisted selection provides more efficient and accurate results than traditional methods. This also allows rapid introduction into or elimination from a particular genetic background of the specific trait or traits associated with the identified genetic marker. In the present case, screening for haplotype alleles conferring increased or desirable beef tenderness may be used to allow the efficient culling of cattle that will not produce beef of a desirable tenderness after electric stimulation, and the selection of cattle that will produce beef with desirable tenderness, as desired.

Genetic assays can be used to obtain information about the genes that influence an important trait, thus facilitating breeding efforts. Factors considered in developing markers for a particular trait include: how many genes influence a trait, where the genes are located on the chromosomes (e.g., near which genetic markers), how much each locus affects the trait, whether the number of copies has an effect (gene dosage), pleiotropy, environmental sensitivity, and epistasis.

A genetic map represents the relative order of genetic markers, and their relative distances from one another, along each chromosome of an organism. During sexual reproduction in higher organisms, the two copies of each chromosome pair align themselves closely with one another. Genetic markers that lie close to one another on the chromosome are seldom recombined, and thus are usually found together in the same progeny individuals. Markers that lie close together show a small percent recombination, and are said to be linked. Markers linked to loci having phenotypic effects are particularly important in that they may be used for selection of individuals having the desired trait.

The identity of a given allele can therefore be determined by identifying nearby genetic markers that are usually co-transmitted with the gene from parent to progeny. This principle applies both to genes with large effects on phenotype (simply inherited traits) and genes with small effects on phenotype. As such, by identifying a marker linked to a particular trait, this will allow direct selection for the linked polymorphism without the need for detecting that particular polymorphism due to genetic linkage between the traits. Those of skill in the art will therefore understand that when genetic assays for a haplotype or marker set forth herein are mentioned, this specifically encompasses detection of genetically linked polymorphisms that are informative for the haplotype or marker allele. Such polymorphisms have predictive power relative to the trait to the extent that they also are linked to the contributing locus for the trait. Such markers thus also have predictive potential for the trait of interest. In this regard, a polymorphism of interest in the present invention may comprise a polymorphism in a haplotype located in a region on bovine chromosome 2 defined by positions −24230649 and −24118992, in a haplotype located in a region on bovine chromosome 2 defined by positions 103881402 and 103950562, or in a haplotype located in a region on bovine chromosome 2 defined by positions 36242459 and 36362623, or a SNP located within or genetically linked to these haplotypes. For example, a polymorphism in accordance with the invention may comprise a polymorphism set forth in Tables 12-14.

Most natural populations of animals are genetically quite different from the classical linkage mapping populations. While linkage mapping populations are commonly derived from two-generation crosses between two parents, many natural populations are derived from multi-generation matings between an assortment of different parents, resulting in a massive reshuffling of genes. Individuals in such populations carry a complex mosaic of genes, derived from a number of different founders of the population. Gene frequencies in the population as a whole may be modified by a natural or artificial selection, or by genetic drift (e.g., chance) in small populations. Given such a complex population with superior average expression of a trait, a breeder might wish to (1) maintain or improve the expression of the trait of interest, while maintaining desirable levels of other traits; and (2) maintain sufficient genetic diversity that rare desirable alleles influencing the trait(s) of interest are not lost before their frequency can be increased by selection.

Genetic assays may find particular utility in maintaining sufficient genetic diversity in a population while maintaining favorable alleles. For example, one might select a fraction of the population based on favorable phenotype (perhaps for several traits—one might readily employ index selection), then apply genetic assays as described herein to this fraction and keep a subset which represent much of the allelic diversity within the population. Strategies for extracting a maximum of desirable phenotypic variation from complex populations remain an important area of breeding strategy. An integrated approach, merging classical phenotypic selection with a genetic marker-based analysis, may aid in extracting valuable genes from heterogeneous populations.

The techniques of the present invention may potentially be used with any bovine, including Bos taurus and Bos indicus cattle. In particular embodiments of the invention, the techniques described herein are specifically applied for selection of beef cattle, as the genetic assays described herein will find utility in maximizing production of animal products, such as meat. As used herein, the term “beef cattle” refers to cattle grown or bred for production of meat or other non-dairy animal products. Therefore, a “head of beef cattle” refers to at least a first bovine animal grown or bred for production of meat or other non-dairy animal products. Examples of breeds of cattle that may be used with the invention include, but are not limited to, Africander, Alberes, Alentejana, American, American White Park, Amerifax, Amrit Mahal, Anatolian Black, Andalusian Black, Andalusian Grey, Angeln, Angus, Ankole, Ankole-Watusi, Argentine Criollo, Asturian Mountain, Asturian Valley, Australian Braford, Australian Lowline, Ba-Bg, Bachaur, Baladi, Barka, Barzona, Bazadais, Beefalo, Beefmaker, Beefmaster, Belarus, Red, Belgian Blue, Belgian Red, Belmont Adaptaur, Belmont Red, Belted Galloway, Bengali, Berrendas, Bh-Bz, Bhagnari, Blanco Orejinegro, Blonde d'Aquitaine, Bonsmara, Boran, Braford, Brahman, Brahmousin, Brangus, Braunvieh, British White, Busa, Cachena, Canary Island, Canchim, Carinthian Blond, Caucasian, Channi, Charbray, Charolais, Chianina, Cholistani, Corriente, Costello con Cuernos, Dajal, Damietta, Dangi, Deoni, Devon, Dexter, Dhanni, Dolafe, Droughtmaster, Dulong, East Anatolian Red, Enderby Island, English Longhorn, Evolene, Fighting Bull, Florida Cracker/Pineywoods, Galician Blond, Galloway, Gaolao, Gascon, Gelbray, Gelbvieh, German Angus, German Red Pied, Gir, Glan, Greek Shorthorn, Guzerat, Hallikar, Hariana, Hays Converter, Hereford, Herens, Highland, Hinterwald, Holando-Argentino, Horro, Hungarian Grey, Indo-Brazilian, Irish Moiled, Israeli Red, Jamaica Black, Jamaica Red, Jaulan, Kangayam, Kankrej, Kazakh, Kenwariya, Kerry, Kherigarh, Khillari, Krishna Valley, Kurdi, Kuri, Limousin, Lincoln Red, Lohani, Luing, Maine Anjou, Malvi, Mandalong, Marchigiana, Masai, Mashona, Mewati, Mirandesa, Mongolian, Morucha, Murboden, Murray Grey, Nagori, N'dama, Nellore, Nguni, Nimari, Ongole, Orma Boran, Oropa, Parthenais, Philippine Native, Polish Red, Polled Hereford, Ponwar, Piedmontese, Pinzgauer, Qinchuan, Ratien Gray, Rath, Rathi, Red Angus, Red Brangus, Red Poll, Retinta, Rojhan, Romagnola, Romosinuano, RX3, Sa-Sg, Sahiwal, Salers, Salorn, Sanhe, Santa Cruz, Santa Gertrudis, San Martinero, Sarabi, Senepol, Sh-Sz, Sharabi, Shorthorn, Simbrah, Simmental, Siri, Slovenian Cika, South Devon, Sussex, Swedish Red Polled, Tarentaise, Telemark, Texas Longhorn, Texon, Tharparkar, Tswana, Tuli, Ukrainian Beef, Ukrainian Grey, Ukrainian Whitehead, Umblachery, Ural Black Pied, Vestland Red Polled, Vosges, Wagyu, Welsh Black, White Caceres, White Park, Xinjiang Brown and Yanbian cattle breeds, as well as animals bred therefrom and related thereto.

### II. Nucleic Acid Detection

Techniques for nucleic acid detection may find use in certain embodiments of the invention. For example, such techniques may find use in scoring individuals for genotypes or in the development of novel markers linked to the major effect locus identified herein.

1. Hybridization

The use of a probe or primer of between 13 and 100 nucleotides, preferably between 17 and 100 nucleotides in length, or in some aspects of the invention up to 1-2 kilobases or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

Accordingly, nucleotide sequences may be used in accordance with the invention for their ability to selectively form duplex molecules with complementary stretches of DNAs and/or RNAs or to provide primers for amplification of DNA or RNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications, lower stringency conditions may be preferred. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M NaCl, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences with the present invention in combination with an appropriate means, such as a label, for determining hybridization. For example, such techniques may be used for scoring of RFLP marker genotype. A wide variety of appropriate indicator means are known in the art, including fluorescent, radioactive, enzymatic or other ligands, such as avidin/biotin, which are capable of being detected. In certain embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples.

In general, it is envisioned that probes or primers will be useful as reagents in solution hybridization, as in PCR, for detection of nucleic acids, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481, and 5,919,626. Other methods of hybridization that may be used in the practice of the present invention are disclosed in U.S. Pat. Nos. 5,849,481, 5,849,486, and 5,851, 772. The relevant portions of these and other references identified in this section of the Specification are incorporated herein by reference.

2. Amplification of Nucleic Acids

Nucleic acids used as a template for amplification may be isolated from cells, tissues or other samples according to standard methodologies. Such embodiments may find particular use with the invention, for example, in the detection of repeat length polymorphisms, such as microsatellite markers. In certain embodiments of the invention, amplification analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form.

Pairs of primers designed to selectively hybridize to nucleic acids are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radio-label or fluorescent label or even via a system using electrical and/or thermal impulse signals. Typically, scoring of polymorphisms as fragment length variants will be done based on the size of the resulting amplification product.

A number of template dependent processes are available to amplify the oligonucleotide sequences present in a given template sample. One of the best known amplification methods is the polymerase chain reaction (referred to as PCR), which is well known in the art.

A reverse transcriptase PCR amplification procedure may be performed to obtain cDNA, which in turn may be scored for polymorphisms. Methods of reverse transcribing RNA into cDNA are well known. Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR and oligonucleotide ligase assay (OLA), disclosed in U.S. Pat. No. 5,912,148, also may be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291, and 5,942,391, GB Application No. 2 202 328, and in PCT Application No. PCT/US89/01025, each of which is incorporated herein by reference in its entirety.

An isothermal amplification method, in which restriction endonucleases and ligases are used to achieve the amplification of target molecules that contain nucleotide 5′-[alpha-thio]-triphosphates in one strand of a restriction site also may be useful in the amplification of nucleic acids in the present invention. Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; Gingeras et al., 1990; PCT Application WO 88/10315, incorporated herein by reference in their entirety). European Application No. 329 822 discloses a nucleic acid amplification process involving cyclically synthesizing single-stranded RNA (ssRNA), single-stranded DNA (ssDNA), and double-stranded DNA (dsDNA), which may be used in accordance with the present invention.

PCT Application WO 89/06700 (incorporated herein by reference in its entirety) discloses a nucleic acid sequence amplification scheme based on the hybridization of a promoter region/primer sequence to a target ssDNA followed by transcription of many RNA copies of the sequence. This scheme is not cyclic, i.e., new templates are not produced from the resultant RNA transcripts. Other amplification methods include “race” and “one-sided PCR.”

3. Detection of Nucleic Acids

Following any amplification, it may be desirable to separate the amplification product from the template and/or the excess primer. In one embodiment, amplification products are separated by agarose, agarose-acrylamide or polyacrylamide gel electrophoresis using standard methods. Separated amplification products may be cut out and eluted from the gel for further manipulation. Using low melting point agarose gels, the separated band may be removed by heating the gel, followed by extraction of the nucleic acid.

Separation of nucleic acids also may be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art. One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413, and 5,935,791, each of which is incorporated herein by reference.

4. Other Assays

Other methods for genetic screening may be used within the scope of the present invention, for example, to detect polymorphisms in genomic DNA, cDNA, and/or RNA samples. Methods used to detect point mutations include denaturing gradient gel electrophoresis (“DGGE”), restriction fragment length polymorphism analysis (“RFLP”), chemical or enzymatic cleavage methods, direct sequencing of target regions amplified by PCR (see above), single-strand conformation polymorphism analysis (“SSCP”) and other methods well known in the art.

One method of screening for point mutations is based on RNase cleavage of base pair mismatches in RNA/DNA or RNA/RNA heteroduplexes. As used herein, the term “mismatch” is defined as a region of one or more unpaired or mispaired nucleotides in a double-stranded RNA/RNA, RNA/DNA or DNA/DNA molecule. This definition thus includes mismatches due to insertion/deletion mutations, as well as single or multiple base point mutations.

U.S. Pat. No. 4,946,773 describes an RNase A mismatch cleavage assay that involves annealing single-stranded DNA or RNA test samples to an RNA probe, and subsequent treatment of the nucleic acid duplexes with RNase A. For the detection of mismatches, the single-stranded products of the RNase A treatment, electrophoretically separated according to size, are compared to similarly treated control duplexes. Samples containing smaller fragments (cleavage products) not seen in the control duplex are scored as positive.

Other investigators have described the use of RNase I in mismatch assays. The use of RNase I for mismatch detection is described in literature from Promega Biotech. Promega markets a kit containing RNase I that is reported to cleave three out of four known mismatches. Others have described using the MutS protein or other DNA-repair enzymes for detection of single-base mismatches.

Alternative methods for detection of deletion, insertion or substitution mutations that may be used in the practice of the present invention are disclosed in U.S. Pat. Nos. 5,849,483, 5,851,770, 5,866,337, 5,925,525, and 5,928, 870, each of which is incorporated herein by reference in its entirety.

5. Kits

All the essential materials and/or reagents required for screening cattle for genetic marker genotype in accordance with the invention may be assembled together in a kit. This generally will comprise a probe or primers designed to hybridize specifically to individual nucleic acids of interest in the practice of the present invention, for example, primer sequences such as those for amplifying a SNP within or genetically linked to a hapblock set forth herein. Also included may be enzymes suitable for amplifying nucleic acids, including various polymerases (reverse transcriptase, Taq, etc.), deoxynucleotides and buffers to provide the necessary reaction mixture for amplification. Such kits also may include enzymes and other reagents suitable for detection of specific nucleic acids or amplification products. Such kits generally will comprise, in suitable means, distinct containers for each individual reagent or enzyme as well as for each probe or primer pair.

### DEFINITIONS

The following definitions are provided to better define the present invention and to guide those of ordinary skill in the art in the practice of the present invention. Unless otherwise noted, terms are to be understood according to conventional usage by those of ordinary skill in the relevant art.

As used herein, the term “population” means a genetically heterogeneous group of animals that share a common parental derivation. A population in accordance with the invention may originate from a single breed or species of beef cattle, or may originate from multiple breeds or species of beef cattle.

As used herein, a “crossbred” or “hybrid” cattle refers to a head of cattle that resulted from a cross of one parent of one breed or species of cattle to another, distinct species or breed of cattle.

As used herein, “SNP” refers to single base positions in DNA at which different alleles, or alternative nucleotides, exist in a population, and are the most common form of genetic variation in the genome. The SNP position (interchangeably referred to herein as SNP, SNP site, SNP locus, SNP marker, biomarker, or marker) is usually preceded by and followed by highly conserved sequences of the allele (e.g., sequences that vary in less than 1/100 or 1/1000 members of the populations). An individual may be homozygous or heterozygous for an allele at each SNP position. In some embodiments, a SNP is referred to as a “cSNP” to denote that the nucleotide sequence containing the SNP is an amino acid coding sequence.

As used herein, references to SNPs and SNP genotypes include individual SNPs and/or haplotypes. Haplotypes may comprise groups of SNPs that are generally inherited together. Haplotypes can have stronger correlations with diseases or other phenotypic effects compared with individual SNPs, and therefore may provide increased diagnostic accuracy. Causative SNPs are those SNPs that produce alterations in gene expression or in the expression, structure, and/or function of a gene product, and therefore are most predictive of a possible clinical phenotype. One such SNP may be present within regions of genes encoding a polypeptide product, i.e. cSNPs. These SNPs may result in an alteration of the amino acid sequence of the polypeptide product (i.e., non-synonymous codon changes) and give rise to the expression of a defective or other variant protein. A SNP may also lead to premature termination of a polypeptide product. As referred to herein, causative SNPs may occur in any genetic region that can ultimately affect the expression, structure, and/or activity of a protein encoded by a nucleic acid. Such genetic regions may include, for example, those involved in transcription, such as SNPs in transcription factor binding domains, promoter regions, or areas involved in transcript processing, such as intron-exon boundaries, which may cause defective splicing, or SNPs in mRNA processing signal sequences such as polyadenylation signal regions. In another embodiment, a SNP may not be a causative SNP but lie in close association with, and therefore segregate with, a sequence producing a trait or disease. In this situation, the presence of a SNP correlates with the presence of, or predisposition to, or an increased chance of the trait and may be useful for diagnostics, predisposition screening, and other uses.

Although the numerical chromosomal position of a SNP may still change upon annotating the current bovine genome, the SNP identification information such as variable alleles and flanking nucleotide sequences assigned to a SNP will remain the same. Genes discussed herein, as well as SNPs listed herein in Tables 12-14 are defined by a genomic position relative to the UMD 3.1 bovine genome assembly (Bos_{—}taurus_UMD_{—}3.1, GenBank assembly ID GCA_{—}000003055.3, Zimin et al., Genome Biology 10:R42, 2009). Those skilled in the art will readily recognize that the analysis of the nucleotides present in one or more SNPs set forth herein in an individual's genome can be done by any method or technique capable of determining nucleotides present in a polymorphic site using the published sequence information to the rs IDs or other SNP identification, such as KGP SNPs, for the SNPs listed herein. The nucleotides present in polymorphisms can be determined from either nucleic acid strand or from both strands.

As used herein, “gene” refers to a nucleic acid molecule that codes for a particular protein, or in certain cases, a functional or structural RNA molecule. Genes that are located on the minus strand of DNA may be indicated by using a negative chromosomal position, although it is generally customary to indicate the position of SNPs relative to the plus strand. For example, a haplotype as discussed herein located in a region on bovine chromosome 2 defined by genome positions −24230649 and −24118992 indicates that the haplotype is present on the minus strand of bovine chromosome 2 defined by those genome positions.

As used herein, a “haplotype” or “haplotype block” or “hapblock” refers to any combination of genetic markers (“alleles”). Such a haplotype may comprise one or more genes or markers conferring a desired trait. In some embodiments, such terms may refer to a set or collection of genes, SNPs, or other types of markers that are associated statistically. A haplotype can include two or more alleles and the length of a genome region including a haplotype may vary from a few hundred bases up to thousands of kilobases or megabases. As it is recognized by those skilled in the art, the same haplotype can be described differently by determining the haplotype defining alleles from different nucleic acid strands. The haplotypes described herein are differentially present in individuals with increased beef tenderness or having an increased chance of producing more tender beef following postmortem electrical stimulation. Therefore, these haplotypes have diagnostic value. Detection of haplotypes can be accomplished by methods known in the art used for detecting nucleotides at polymorphic sites. The haplotypes described herein, e.g., having markers or SNPs set forth herein, are found more frequently in cattle that will produce beef with increased tenderness than in individuals that produce beef with less tenderness. Alternatively, certain haplotypes may be found more frequently in cattle that do not produce beef with increased tenderness following postmortem electrical stimulation.

As used herein, an “allele” refers to one of two or more alternative forms of a genomic sequence at a given locus on a chromosome.

A “Quantitative Trait Locus (QTL)” is a chromosomal location that encodes for alleles that affect the expressivity of a phenotype.

As used herein, a “marker” means a detectable characteristic that can be used to discriminate between organisms. Examples of such characteristics include, but are not limited to, genetic markers, biochemical markers, metabolites, morphological characteristics, and agronomic characteristics.

As used herein, the term “phenotype” means the detectable characteristics of a cell or organism that can be influenced by gene expression.

As used herein, the term “genotype” means the specific allelic makeup of an animal.

As used herein, the term “introgressed” or “introduced.” when used in reference to a genetic locus or haplotype, refers to a genetic locus that has been introduced into a new genetic background. Introgression of a genetic locus can thus be achieved through breeding methods.

As used herein, the term “linked,” when used in the context of nucleic acid markers and/or genomic regions, means that the markers and/or genomic regions are located on the same linkage group or chromosome such that they tend to segregate together at meiosis.

As used herein, an “estimated breeding value” is a statistical numerical prediction of the relative genetic value of a particular individual for breeding. In one embodiment of the invention, an individual may be selected for breeding based upon its estimated breeding value. In particular, an individual with a desirable estimated breeding value may be selected. In certain embodiments, a desirable estimated breeding value may refer to an estimated breeding value that is greater than the average estimated breeding value of the population of individuals being selected from. For instance, a desired estimated breeding value may be within the top 50%, 40%, 30%, 25%, 20%, 15%, 10%, 5%, 4%, 3%, 2%, 1% of the population the individual is selected from.

As used herein, the term “denoting” when used in reference to a genotype refers to any method whereby an animal or individual is indicated to have a certain genotype. This includes any means of identification of an individual having a certain genotype. Indication of a certain genotype may include, but is not limited to, any entry into any type of written or electronic medium or database whereby the individual's genotype is provided. Indications of a certain genotype may also include, but are not limited to, any method where an individual is physically marked or tagged. Illustrative examples of physical marking or tags useful in the invention include, but are not limited to, a barcode, a radio-frequency identification (RFID), a label, or the like.

As used herein, the singular forms “a,” “an,” “and,” and “the” include plural referents unless the context clearly dictates otherwise. Thus, for example, reference to “a SNP” includes a plurality of SNPs (i.e., at least one SNP), reference to “a gene” includes a plurality of genes (i.e., at least one gene), and so forth.

### EXAMPLES

The following disclosed embodiments are merely representative of the invention which may be embodied in various forms. Thus, specific structural, functional, and procedural details disclosed in the following examples are not to be interpreted as limiting.

### Example 1

### Herd Structure

All samples used in this project came from a herd established in McGregor, Tex. in 2003 as part of the McGregor Genomics project. The McGregor Genomics project was established to identify genetic factors that contribute to beef cattle productivity. This project enables QTL analyses for economically important traits in cattle including feed efficiency, carcass and meat traits, and behavior and female reproductive traits (Gill, 2004). The herd consists of 13 full-sibling F2 embryos transfer families derived over a 5 year time period from 13 Nellore-Angus F1 females and 4 Nellore-Angus F1 males (Sanders, 2008). Progeny were created by a mixture of natural service and embryo transfer. Only steers in the F2 population produced by embryo-transfer were used in this study.

Steers were fed as yearlings and harvested at approximately 18 mo of age at the Texas A&M University Rosenthal Meat Science and Technology Center in College Station, Tex. All carcasses were split laterally along the spine during processing. The right side was subjected to electrical stimulation, and as a control the left side was not electrically stimulated. Muscle samples were taken from between the 12^{th} and 13^{th} ribs, as this is the location for USDA grade assessment. Warner-Bratzler shear force (WBSF) and sensory panel evaluation were performed in the Kleberg Center (Metteauer, 2009). WBSF measures the total force necessary to shear a piece of cooked meat under controlled circumstances and reduces the subjectivity inherent to consumer panel ratings. WBSF is an objective measure of final tenderness that takes in to account all factors that affect this trait.

### Example 2

### Sample Selection

The samples used in this study were obtained from F2 Nellore-Angus steers produced by embryo transfer. Females were kept for breeding and thus were excluded from any meat quality studies. This would have been done regardless as gender is already an established variable that affects tenderness (Baird, 2007). All animals used in this study were produced by embryo transfer (ET). Shortly postmortem (within 60 min after captive bolt stunning), muscle samples from the Sternomandibularis muscle were collected and flash frozen in liquid nitrogen to preserve RNA integrity. Samples were transferred to a −80° C. freezer for long term storage. All muscle samples used in this experiment were collected prior to electrical stimulation (ES). It is unknown what effect ES has on mRNA and protein expression in the short term postmortem. This means that every animal had two recorded shear force values, one for the half that was not subjected to electrical stimulation (NES) and the other for the half that was subjected to electrical stimulation (ES). For every animal only one muscle sample was collected prior to ES.

To further reduce the total number of variables, a statistical model was created to address factors known to affect tenderness. A mixed-model in SPSS v16.0 was utilized to produce this model. Fixed class effects were: calving group (year and season) and slaughter date. Random class effects were: sire and family nested within sire. Total age at time of slaughter, measured in days, was factored in as a linear covariate. This same process was performed separately for both ES and NES WBSF values. Residual differences were used to assign tenderness groups. Those with positive residuals (actual WBSF greater than model predicted) were designated as “high” and those with negative residuals (actual WBSF lower than model predicted) were deemed “low.”

Twelve extremes of tenderness from each tail of the distribution were selected using ES residuals, those that were tougher than predicted (“high”) and those that were more tender than predicted (“low”). This produced a total of 24 samples for the ES residual group. Additionally, 12 extremes were selected using the NES residual values at either end. A total of 24 unique samples were selected from the NES group. In this way, a total of 48 samples were selected for microarray analysis. All samples were collected prior to electrical stimulation for all animals. The ES grouping is therefore based on WBSF measurements made following ES. The muscle sample used for gene and protein expression assays, however, was not subject to ES. This was necessary to reduce variability due to currently unknown effects ES would have on muscle gene and protein expression. One sample, 7203, was absent from the muscle sample stocks. It was removed from further analysis

### Example 3

### Tissue Collection

Approximately 1 g muscle was collected shortly postmortem prior to ES (less than 1 h post-exsanguination) from the Sternomandibularis. The sample was flash frozen in liquid nitrogen to prevent mRNA degradation. Samples were stored at −80° C.

### Example 4

### RNA Extraction

RNA was extracted from approximately 100 to 200 mg of whole muscle tissue with TRI Reagent® (Molecular Research Center, Cincinnati, Ohio) and 1-bromo-3-chloropropane (Molecular Research Center). RNA was precipitated with isopropanol (Sigma Aldrich, St. Louis, Mo.), washed with 70% ethanol (Sigma Aldrich), and reconstituted in 50 μL nuclease-free water (Invitrogen, Carlsbad, Calif.). RNA was tested for quality on an Agilent 2100 series Bioanalyzer (Agilent Technologies, Palo Alto, Calif.) according to the manufacturer's protocol. Samples with an RNA integrity number (RIN) >8.0 were treated with DNase (Invitrogen) and column-purified via RNeasy Mini kit (Qiagen, Valencia, Calif.). Samples were stored at −80° C. until use.

### Example 5

### Microarray

An initial microarray study was used to identify candidate genes for follow up study. Samples were selected based on ES residuals for 24 (12 high and 12 low) animals and on NES residuals for 24 animals (12 high and 12 low) for a total of 48 samples.

A single color 44K bovine array (B. taurus (Bovine) Oligo Microarray v2 Agilent 4X44K GPL11649) was used according to manufacturer's instructions. The single color array was chosen as natural variation in gene expression would have made pairing samples on a 2 color array impractical. Spike-in procedure was performed, after which a master mix of 15.3 μL nuclease free water, 20.0 μL 4× transcription buffer, 6.0 μL 0.1 M DTT, 8.0 μL NTP mix, 6.4 μL 50% PEG, 0.5 μL RNaseOUT, 0.6 μL inorganic pyrophosphatase, 0.8 μL T7 RNA polymerase, and 2.4 μL cyanine 3-CTP or cyanine 5-CTP was added and incubated at 40° C. for 2 h. Labeled cRNA was hybridized to bovine 4X44K stock arrays (Agilent array #015344). Fragmentation was performed by combining 825 ng cRNA, 11 μL 10× blocking agent, 2.2 μL 25× fragmentation buffer in a total volume of 55 μL per sample and heating 30 min at 60° C. To end fragmentation a total of 55 μL GEx Hybridization buffer was added for a total volume of 110 μL per sample. A total of 100 μL labeled sample was applied to each microarray and were hybridized in at 65° C. for 17 h in a hybridization chamber. Arrays were washed and scanned immediately after this time. Normalization and quality control were performed using embedded functions in the Genespring GX v11.0.2 software.

Quantile normalization was used to reduce variability between arrays. Array data were subjected to baseline normalization to the median expression level and had a minimum threshold of 1.0 expression all using standard quality control options built in to the Genespring software. Quality control on arrays was performed via correlation plots. If expression between an array and all other arrays within a population dropped below a correlation of 0.9 it was deemed unreliable. Five arrays were found to be outliers using this method (7151, 7227, 7606, 7732, and 8148). These samples were removed from further consideration in this project. In order to maintain a sufficient population size for statistical analysis groups were recalculated at this point to ensure that the ES group had a total of 12 extremes at each end.

Importantly, reassignment of animals was performed before any statistical analyses were performed. Adjusting labels to account for removed samples did not result in any significant change to population metrics (Table 1).

TABLE 1
Original and revised population statistics for samples used in microarray
analysis

| | | | | Predicted | Residuals |
|---|---|---|---|---|---|
| | Group (ES) | N | WBSF (ES)^{a} | WBSF (ES)^{b} | (ES)^{c} |
| Revised | High | 12 | 4.14 | 2.88 | 1.1 |
| | Low | 12 | 2.11 | 2.79 | −0.79 |
| Initial | High | 12 | 4.14 | 2.83 | 1.06 |
| | Low | 12 | 2.11 | 2.82 | −0.71 |

^{a}WBSF (ES) = observed Warner-Bratzler shear force on carcass half subjected to electrical stimulation (ES).
^{b}Predicted WBSF (ES) = predicted Warner-Bratzler shear force on carcass half subjected to ES based on model predictions.
^{c}Residuals (ES) = difference between observed ES and predicted ES shear force values.

All further analysis is based on these 42 samples (5 removed as outliers based on microarray results and 1 where the sample was missing from the stocks).

Probes were filtered based on flags. Flags for “population outlier”, “saturated”, “not positive or significant”, “not uniform”, and “not above background” were assigned automatically by Agilent Extraction Software v9.5. A further explanation of the calculations used to assign these flags is available in the Agilent Extraction Software v9.5 manual, downloaded from the following uniform resource locator.

For this experiment all flags except “not uniform” and “not above background” were assigned the label “marginal”. Probes flagged as “not uniform” or “not above background” were deemed failed or aberrant and were labeled “absent” and were not used further. A probe without flags was given the default label “present”. As a cut off, 8 out of 24 probes for any given point must have a value of “present” or “marginal” to be considered for statistical analysis.

The Mann-Whitney unpaired test was used for both ES and NON in separate analyses. A False Discovery Rate (FDR) was not used as these were these samples failed the assumption of independence. A non-parametric test was used to avoid relying on the a priori assumption of a normal distribution of gene expression within this population selected for extremes. A cut off of P<0.05 and fold change 1.4 fold or higher between groups was employed as the thresholds to generate lists of probes for subsequent pathways analysis.

A total of 43,713 unique probes were on each array. Of these 32,900 passed the quality control for acceptable levels of present or marginal probes. Filtering by significance and fold change reduced this to 1,046 unique probes. Of these, 867 had usable identifiers (gene annotations that could be recognized by the DAVID software). This is presented in Table 2.

TABLE 2
Numbers of unique probes at various stages of the microarray analysis

| | Total Number of | % of |
|---|---|---|
| | Probes | Total |
| Total Probes on array | 43,713 | 100.00 |
| Passed quality control^{a} | 32,900 | 75.26 |
| 1.4 Fold expression difference^{b} | 1,046 | 2.45 |
| Usable identifiers | 867 | 1.98 |

^{a}Quality control based on flags
^{b}P ≦ 0.05.

Bovine microarray analysis was conducted on skeletal muscle RNA samples from 48 steers. Samples represented 4 tenderness groups in which loin steaks from the carcasses were evaluated for Warner-Bratzler shear force. For both the ES and NES carcass halves, model predicted WBSF values for both, residuals, and initial classifications for the ES or NES group is presented in Table 3. It is worth noting that some samples could be classified in an extreme group in both the ES and NES. Following the removal of unusable outliers the groups were reassigned. These data are presented in Table 4. All subsequent references to ES residual tenderness groups come from Table 4.

The random effects of sire (P=0.03) and family nested within sire (P=0.02) as well as the fixed effects of calving group (P<0.01) and slaughter date (P<0.01) were significant components of the model, developed and described in the methods section, and were retained.

TABLE 3
Tenderness and classification information on 48 animals that were used in this study

| | | Predicted | | | | Predicted | | |
|---|---|---|---|---|---|---|---|---|
| | WBSF, | WBSF, | Residuals | Group | WBSF, | WBSF, | Residuals | Group |
| ID | kg (ES)^{a} | kg (ES)^{b} | (ES)^{c} | (ES) | kg (NES)^{d} | kg (NES)^{e} | (NES)^{f} | (NES) |
| 7009 | 4.59 | 2.82 | 1.72 | High | 3.00 | 3.36 | −0.36 | |
| 7126 | 3.48 | 2.40 | 1.16 | High | 3.44 | 3.58 | −0.14 | |
| 7232 | 4.61 | 2.64 | 1.95 | High | 3.88 | 3.81 | 0.07 | |
| 7715 | 4.06 | 2.99 | 1.04 | High | 4.75 | 3.98 | 0.77 | |
| 7730 | 4.23 | 3.09 | 1.11 | High | 3.17 | 3.98 | −0.81 | |
| 7732 | 3.86 | 2.85 | 0.92 | High | 4.68 | 3.85 | 0.83 | |
| 8010 | 3.78 | 2.64 | 1.08 | High | 4.00 | 3.67 | 0.33 | |
| 8050 | 4.90 | 3.67 | 1.18 | High | 5.38 | 4.50 | 0.88 | |
| 8113 | 3.75 | 2.66 | 0.97 | High | 3.54 | 3.68 | −0.14 | |
| 8146 | 3.97 | 2.79 | 1.04 | High | 3.34 | 3.75 | −0.41 | |
| 8157 | 4.67 | 3.53 | 0.94 | High | 5.36 | 6.13 | −0.77 | |
| 8420 | 4.32 | 3.49 | 0.93 | High | 5.61 | 4.64 | 0.97 | |
| 7005 | 2.18 | 2.82 | −0.68 | Low | 3.56 | 3.36 | 0.20 | |
| 7115 | 1.72 | 2.83 | −0.91 | Low | 3.62 | 3.07 | 0.55 | |
| 7123 | 1.64 | 2.40 | −0.68 | Low | 2.39 | 3.58 | −1.19 | |
| 7142 | 2.52 | 3.53 | −0.97 | Low | 6.81 | 6.13 | 0.68 | |
| 7203 | 2.42 | 2.96 | −0.58 | Low | 3.39 | 4.34 | −0.95 | |
| 7606 | 1.96 | 2.82 | −0.74 | Low | 2.30 | 3.36 | −1.06 | |
| 7728 | 2.23 | 2.79 | −0.58 | Low | 5.12 | 3.75 | 1.37 | |
| 7731 | 1.90 | 2.79 | −0.91 | Low | 3.18 | 3.75 | −0.57 | |
| 8004 | 2.04 | 2.64 | −0.65 | Low | 2.11 | 3.67 | −1.56 | |
| 8115 | 2.28 | 2.83 | −0.60 | Low | 3.07 | 3.07 | 0.00 | |
| 8133 | 1.61 | 2.40 | −0.95 | Low | 3.54 | 3.58 | −0.04 | |
| 8419 | 2.41 | 3.49 | −0.98 | Low | 4.70 | 4.64 | 0.06 | |
| 7021 | 3.68 | 2.79 | 0.90 | | 6.15 | 3.75 | 2.40 | High |
| 7127 | 2.43 | 2.39 | 0.16 | | 5.50 | 3.39 | 2.11 | High |
| 7151 | 2.77 | 2.95 | −0.11 | | 7.17 | 4.92 | 2.25 | High |
| 7215 | 2.77 | 2.45 | 0.36 | | 7.06 | 3.98 | 3.08 | High |
| 7227 | 2.75 | 2.64 | 0.09 | | 5.74 | 3.81 | 1.93 | High |
| 7303 | 2.57 | 2.96 | −0.36 | | 6.37 | 4.34 | 2.03 | High |
| 7742 | 2.72 | 2.66 | 0.00 | | 5.22 | 3.32 | 1.90 | High |
| 8019 | 2.88 | 2.99 | −0.14 | | 6.89 | 3.98 | 2.91 | High |
| 8035 | 2.50 | 2.39 | 0.10 | | 5.14 | 3.39 | 1.75 | High |
| 8125 | 2.47 | 2.21 | 0.17 | | 4.43 | 2.71 | 1.72 | High |
| 8148 | 2.76 | 2.64 | −0.07 | | 5.86 | 3.81 | 2.05 | High |
| 8159 | 2.81 | 2.59 | 0.16 | | 7.78 | 6.10 | 1.68 | High |
| 7101 | 4.00 | 2.96 | 1.07 | | 2.72 | 4.34 | −1.62 | Low |
| 7112 | 2.61 | 2.45 | 0.28 | | 2.66 | 3.98 | −1.32 | Low |
| 7518 | 2.40 | 2.99 | −0.39 | | 2.41 | 3.98 | −1.57 | Low |
| 7736 | 2.37 | 2.59 | −0.16 | | 4.43 | 6.10 | −1.67 | Low |
| 7738 | 3.23 | 2.78 | 0.43 | | 2.67 | 3.98 | −1.31 | Low |
| 8053 | 3.59 | 3.67 | −0.13 | | 3.13 | 4.50 | −1.37 | Low |
| 8156 | 4.40 | 2.95 | 1.27 | | 3.47 | 4.92 | −1.45 | Low |
| 8208 | 1.47 | 2.64 | −1.10 | | 2.58 | 3.81 | −1.23 | Low |
| 8303 | 2.36 | 2.45 | 0.01 | | 2.54 | 3.98 | −1.44 | Low |
| 8306 | 2.70 | 2.99 | −0.20 | | 2.16 | 3.98 | −1.82 | Low |
| 8314 | 2.27 | 2.68 | −0.34 | | 2.20 | 3.65 | −1.45 | Low |
| 8328 | 2.33 | 2.64 | −0.26 | | 1.97 | 3.81 | −1.84 | Low |

^{a}WBSF (ES) = observed Warner-Bratzler shear force on carcass half subjected to electrical stimulation (ES).
^{b}Predicted WBSF (ES) = predicted Warner-Bratzler shear force on carcass half subjected to ES based on model predictions.
^{c}Residuals (ES) = difference between observed ES and predicted ES shear force values.
^{d}WBSF (NES) = observed Warner-Bratzler shear force on carcass half not subjected to electrical stimulation (NES).
^{e}Predicted WBSF (NES) = predicted Warner-Bratzler shear force on NES carcass half not subjected to ES based on model predictions.
^{f}Residuals (NES) = difference between observed NES and predicted NES shear force values.

A total of 48 single-color microarray hybridizations were conducted on catalog bovine arrays (Agilent). Six hybridizations did not pass quality control screening and were removed. These 6 samples were removed from all subsequent analysis. The amended list is presented in Table 4.

TABLE 4
Tenderness and classification information on revised list of 42 animals used for the tenderness
study following removal of statistical outliers as determined by microarray analysis

| | | Predicted | | | | Predicted | | |
|---|---|---|---|---|---|---|---|---|
| | WBSF, | WBSF, | Residuals | Group | WBSF, | WBSF, | Residuals | Group |
| ID | kg (ES)^{a} | kg (ES)^{b} | (ES)^{c} | (ES) | kg (NES)^{d} | kg (NES)^{e} | (NES)^{f} | (NES) |
| 7101 | 4.00 | 2.96 | 1.07 | High | 2.72 | 4.34 | −1.65 | Low |
| 8156 | 4.40 | 2.95 | 1.27 | High | 3.47 | 4.92 | −1.34 | Low |
| 7009 | 4.59 | 2.82 | 1.72 | High | 3.00 | 3.36 | −0.36 | |
| 7126 | 3.48 | 2.40 | 1.16 | High | 3.44 | 3.58 | −0.17 | |
| 7232 | 4.61 | 2.64 | 1.95 | High | 3.88 | 3.81 | 0.06 | |
| 7715 | 4.06 | 2.99 | 1.04 | High | 4.75 | 3.98 | 0.67 | |
| 7730 | 4.23 | 3.09 | 1.11 | High | 3.17 | 3.98 | −0.87 | |
| 8010 | 3.78 | 2.64 | 1.08 | High | 4.00 | 3.67 | 0.33 | |
| 8050 | 4.90 | 3.67 | 1.18 | High | 5.38 | 4.50 | 0.82 | |
| 8113 | 3.75 | 2.66 | 0.97 | High | 3.54 | 3.68 | −0.03 | |
| 8146 | 3.97 | 2.79 | 1.04 | High | 3.34 | 3.75 | −0.31 | |
| 8157 | 4.67 | 3.53 | 0.94 | High | 5.36 | 6.13 | −0.68 | |
| 8004 | 2.04 | 2.64 | −0.65 | Low | 2.11 | 3.67 | −1.56 | Low |
| 8314 | 2.27 | 2.68 | −0.34 | Low | 2.20 | 3.65 | −1.39 | Low |
| 7005 | 2.18 | 2.82 | −0.68 | Low | 3.56 | 3.36 | 0.20 | |
| 7115 | 1.72 | 2.83 | −0.91 | Low | 3.62 | 3.07 | 0.40 | |
| 7123 | 1.64 | 2.40 | −0.68 | Low | 2.39 | 3.58 | −1.22 | |
| 7142 | 2.52 | 3.53 | −0.97 | Low | 6.81 | 6.13 | 0.59 | |
| 7728 | 2.23 | 2.79 | −0.58 | Low | 5.12 | 3.75 | 1.32 | |
| 7731 | 1.90 | 2.79 | −0.91 | Low | 3.18 | 3.75 | −0.63 | |
| 8115 | 2.28 | 2.83 | −0.60 | Low | 3.07 | 3.07 | 0.03 | |
| 8133 | 1.61 | 2.40 | −0.95 | Low | 3.54 | 3.58 | 0.11 | |
| 8208 | 1.47 | 2.64 | −1.10 | Low | 2.58 | 3.81 | −1.24 | |
| 8419 | 2.41 | 3.49 | −0.98 | Low | 4.70 | 4.64 | 0.00 | |
| 7021 | 3.68 | 2.79 | 0.90 | | 6.15 | 3.75 | 2.34 | High |
| 7127 | 2.43 | 2.39 | 0.16 | | 5.50 | 3.39 | 2.11 | High |
| 7151 | 2.77 | 2.95 | −0.11 | | 7.17 | 4.92 | 2.18 | High |
| 7215 | 2.77 | 2.45 | 0.36 | | 7.06 | 3.98 | 3.07 | High |
| 7227 | 2.75 | 2.64 | 0.09 | | 5.74 | 3.81 | 1.92 | High |
| 7303 | 2.57 | 2.96 | −0.36 | | 6.37 | 4.34 | 2.06 | High |
| 7742 | 2.72 | 2.66 | 0.00 | | 5.22 | 3.32 | 1.79 | High |
| 8019 | 2.88 | 2.99 | −0.14 | | 6.89 | 3.98 | 2.80 | High |
| 8035 | 2.50 | 2.39 | 0.10 | | 5.14 | 3.39 | 1.75 | High |
| 8125 | 2.47 | 2.21 | 0.17 | | 4.43 | 2.71 | 1.76 | High |
| 8148 | 2.76 | 2.64 | −0.07 | | 5.86 | 3.81 | 2.15 | High |
| 8159 | 2.81 | 2.59 | 0.16 | | 7.78 | 6.10 | 1.75 | High |
| 7112 | 2.61 | 2.45 | 0.28 | | 2.66 | 3.98 | −1.39 | Low |
| 7518 | 2.40 | 2.99 | −0.39 | | 2.41 | 3.98 | −1.50 | Low |
| 7736 | 2.37 | 2.59 | −0.16 | | 4.43 | 6.10 | −1.75 | Low |
| 7738 | 3.23 | 2.78 | 0.43 | | 2.67 | 3.98 | −1.32 | Low |
| 8053 | 3.59 | 3.67 | −0.13 | | 3.13 | 4.50 | −1.43 | Low |
| 8303 | 2.36 | 2.45 | 0.01 | | 2.54 | 3.98 | −1.32 | Low |
| 8306 | 2.70 | 2.99 | −0.20 | | 2.16 | 3.98 | −1.74 | Low |
| 8328 | 2.33 | 2.64 | −0.26 | | 1.97 | 3.81 | −1.72 | Low |
| 7203 | 2.42 | 2.96 | −0.58 | | 3.39 | 4.34 | −0.91 | |
| 7606 | 1.96 | 2.82 | −0.74 | | 2.30 | 3.36 | −1.06 | |
| 7732 | 3.86 | 2.85 | 0.92 | | 4.68 | 3.85 | 0.81 | |
| 8420 | 4.32 | 3.49 | 0.93 | | 5.61 | 4.64 | 0.92 | |

^{a}WBSF (ES) = observed Warner-Bratzler shear force on carcass half subjected to electrical stimulation (ES).
^{b}Predicted WBSF (ES) = predicted Warner-Bratzler shear force on carcass half subjected to ES based on model predictions.
^{c}Residuals (ES) = difference between observed ES and predicted ES shear force values.
^{d}WBSF (NES) = observed Warner-Bratzler shear force on carcass half not subjected to electrical stimulation (NES).
^{e}Predicted WBSF (NES) = predicted Warner-Bratzler shear force on NES carcass half not subjected to ES based on model predictions.
^{f}Residuals (NES) = difference between observed NES and predicted NES shear force values.

A non-parametric Mann-Whitney test with no FDR was used to determine significance between ES and NES tenderness groups. A total of 32,900 genes passed quality control for both ES and NES tenderness groups. Within the ES tenderness group 1,937 probes that were significantly different between groups. From those results 1,046 probes had a 1.4 fold or greater difference in expression levels between ES tenderness groups.

### Example 6

### Pathway Analysis

A pathway analysis was performed using DAVID Bioinformatics Resources 6.7. A total of 1,071 genes were differentially expressed from the microarray at a significant level. Of those, 867 were associated with known genes. DAVID software was able to reliably identify 752 of those gene symbols in the bovine specific pathway set. A minimum count of 2 and cutoff of P<0.1 was used to determine if a KEGG pathway was significantly enriched.

Gene Ontology (GO) analysis was also performed within the DAVID software using the same data set that was used for pathway analysis. The GO-fat category was used with the default ease setting of 0.1 and minimum count of 2.

A pathway analysis of microarray results was performed using DAVID Bioinformatics Resources 6.7 (http://david.abcc.ncifcrf.gov/). A total of 1,046 genes were differentially expressed (1.4 fold or greater) from the microarray at a significant level. Of those, 867 were associated with known genes. DAVID software was used to place 752 of these genes in the bovine specific pathway sets. Pathway analysis revealed a total of 15 significantly enriched pathways (Table 5). Focal adhesion, cell adhesion, and ECM-receptor pathways are all closely related functionally and were found to be significantly enriched.

TABLE 5
Significantly enriched pathways based on DAVID analysis

| Term | Count | % | P-Value |
|---|---|---|---|
| Pathways in cancer | 22 | 0.3 | 0.08 |
| Regulation of actin cytoskeleton | 18 | 0.2 | 0.01 |
| Focal adhesion | 15 | 0.2 | 0.07 |
| Cell adhesion molecules (CAMs) | 14 | 0.2 | 0.01 |
| T cell receptor signaling pathway | 11 | 0.1 | 0.04 |
| Axon guidance | 11 | 0.1 | 0.06 |
| Tight junction | 11 | 0.1 | 0.09 |
| ECM-receptor interaction | 9 | 0.1 | 0.03 |
| B cell receptor signaling pathway | 8 | 0.1 | 0.06 |
| Autoimmune thyroid disease | 7 | 0.1 | 0.02 |
| Homologous recombination | 6 | 0.1 | 0.01 |
| Type I diabetes mellitus | 6 | 0.1 | 0.04 |
| Intestinal immune network for IgA production | 6 | 0.1 | 0.09 |
| Fructose and mannose metabolism | 5 | 0.1 | 0.06 |
| Allograft rejection | 5 | 0.1 | 0.09 |

Enrichment is determined by 752 genes recognized by DAVID Bioinformatics overlaid on KEGG bovine pathways.

The pathways in cancer subset was not chosen due to the redundant role of many genes enriched in it that are important in normal muscle physiology and regulation.

GO analysis performed on the same data gave similar results with categories relating to cell adhesion, motility, and migration being highly enriched (Table 6).

TABLE 6
GO analysis based on microarray expression data

| Term | Count | % | P-Value |
|---|---|---|---|
| Cell adhesion | 26 | 0.3 | 0.002 |
| Biological adhesion | 26 | 0.3 | 0.002 |
| Cell motion | 18 | 0.2 | <0.001 |
| Cell migration | 16 | 0.2 | <0.001 |
| Localization of cell | 16 | 0.2 | <0.001 |
| Cell motility | 16 | 0.2 | <0.001 |
| Hexose metabolic process | 15 | 0.2 | <0.001 |
| Monosaccharide metabolic process | 15 | 0.2 | 0.001 |
| Leukocyte activation | 14 | 0.2 | 0.001 |
| Cell activation | 14 | 0.2 | 0.003 |
| Lymphocyte activation | 13 | 0.2 | 0.001 |
| Glucose metabolic process | 11 | 0.1 | 0.007 |
| Antigen processing and presentation | 10 | 0.1 | 0.010 |
| Leukocyte differentiation | 9 | 0.1 | 0.005 |
| Skeletal system morphogenesis | 8 | 0.1 | 0.006 |
| Antigen processing and presentation of peptide or | 6 | 0.1 | 0.001 |
| polysaccharide antigen via MHC class II | | | |

GO analysis performed by DAVID bioinformatics based on 752 genes.

DAVID analysis revealed 15 significantly enriched pathways in the bovine set. The focal adhesion pathway overlapped with the ECM-receptor and regulation of actin cytoskeleton pathways. Additionally focal adhesion is a factor in muscle cell adhesion, cell motion and adhesion, and muscle assembly. F or these reasons genes in the focal adhesion pathway were the focus of follow up qRT-PCR verification.

### Example 7

### PCR

To verify expression data obtained from microarray analysis quantitative real-time reverse-transcriptase PCR (qRT-PCR) was used. Quantitative real-time RT-PCR can be used to validate expression results obtained from large scale gene expression assays such as microarrays. Messenger RNA extracted for the microarray assay was reverse transcribed using oligo(dT) primers Integrated DNA Technologies (Coralville, Iowa) and the High-capacity cDNA Reverse Transcription Kit (Applied Biosystems, Inc.) with a starting quantity of 2 μg mRNA per 40 μL reaction.

The population used in this study was the same as before with the same 5 samples that were found to be unreliable in the microarray set removed (7151, 7227, 7606, 7732, and 8148) for a total of 42 samples remaining. Because one goal of this step was to verify and add to the results of the microarray study, any samples not being used previously were considered undesirable for follow-up studies.

A total of 0.6 μg of RNA were used per reaction well for cDNA generation. Each sample was amplified in duplicate in batches of 20 μL and recombined following amplification; the protocol used was in accordance with manufacturer specifications with no deviations. A 20 μL reaction was prepared as follows: 9.2 μL of RNA quality water, 2.0 μL 10× RT buffer stock, 2.0 μL oligo(dT) primers, 0.8 μL dNTP, 1.0 μL RT enzyme, and 5.0 μL diluted RNA sample. These were then amplified using an ABI2720 thermal cycler following the manufacturers protocol; 25° C. for 10 min, 37° C. for 2 h, and 70° C. for 2 min.

Following amplification samples were diluted 1:5 in 25 ng/μL yeast tRNA following lab protocol. Working stocks were stored at 4° C. and master aliquots were stored at −20° C.

Primers were chosen based on results from the microarray and pathway analysis as well as from the literature. Primer design followed lab protocol using Oligo Primer Analysis Software v6.71 by Molecular Biology Insights (Cascade, Colo.). Primers were selected where at least one primer within a pair covered an exon junction as a precaution against amplifying genomic DNA. Additionally, they were selected to have a melting temperature (Tm) close to 60° C., an approximate length of 20 base pairs, roughly equal balance of nucleotides not skewed towards G/Cs or A/Ts, and a dimerization of AG no less than −4.0 at any point particularly at the 3′ end. Potential primers were then analyzed using Basic Local Alignment Search Tool (BLAST) provided freely by NCBI. A search of the Bos taurus database (taxid: 9913) for highly similar sequences (megablast) was employed to determine if the primer sequence would amplify non-specifically. Primers were ordered from Integrated DNA Technologies (Coralville, Iowa) and diluted in RNA-grade water to a working concentration of 10 μM. Genes that were candidates for primer design consisted of those selected from the pathway analysis, those believed to be significant from the literature although not found to be significant in our assays, and potential control genes selected from the literature. Primer pairs used are shown in Table 7.

TABLE 7
Primers used for qRT-PCR. SEQ ID NOs are shown in parentheses.

| Primer | Forward (SEQ ID NO) | Reverse (SEQ ID NO) |
|---|---|---|
| 18S | TGCCGGAGTCTCGTTCGT (1) | GGTGCATGGCCGTTCTTAGT (2) |
| ACTA1 | CGACGGTCAGGTCATCA (3) | TGCTGTTGTAGGTGGTCTCAT (4) |
| ACTN1 | AAGATGGAGGAGATTGGAAGGAT (5) | GGCTTGTAGTTGACGATGCTCT (6) |
| ACTN2 | GGTCTTTGACAACAAGCA (7) | TGATGGTTCTGGCGATA (8) |
| ACTN3 | CGGGAGACAAGAACTACATCA (9) | CGTAGAGGGCACTGGAGAA (10) |
| ACTN4 | AGCAGAGCAAACAACAGTCCAA (11) | CCTCCAGCGTCCCATTCAT (12) |
| B2M | AGTAAGCCGCAGTGGAGGT (13) | CGCAAAACACCCTGAAGACT (14) |
| C4A | GCCGCCTTCAGTTGGAA (15) | GGTCTCCCTTGAGGTCGTAAGTG (16) |
| CAPN1 | GACCATAGGCTTCGCTGTCT (17) | AGGTTGATGAACTGCTCGGA (18) |
| CAPN2 | CGACTGGAGACACTGTTCAGGA (19) | CTTCAGGCAGATTGGTTATCACTT (20) |
| CAST | GCTGTCGTCTCTGAAGTGGTT (21) | GGCATCGTCAAGTTCTTTGTTGT (22) |
| COL1A2 | GGGCAACAGCAGATTCACTTACA (23) | TCAAGGATAGGCAGGCGAGAT (24) |
| COL6A1 | TGAAAATGTGCTCTTGCTGTGAGT (25) | AATGACCTTGACGATGAAGTCCTT (26) |
| COX7C | TGCAGCCGCCATTTCTTC (27) | TAGCGCTGTTGGACGCTCTA (28) |
| CTSF | GCTGGAGACGGAGGATGA (29) | CACTGAGTCGTTGATGTAGACCTT (30) |
| FLNA | CTGACCAAGACTGCCACCAT (31) | ATGTTGTTGCCTGCTTTGCT (32) |
| FLNB | CGTCTCCACCAAGTTCGCT (33) | TCACAGATGCTGCCAACAGT (34) |
| FLNC | AAGCAGGCACCAATATGATGAT (35) | CCACTTGACGATGAGGATGTAGT (36) |
| IPO7 | CTTCCCTAATAATGTTGAACCAGTTAC (37) | GAACACACATCTTTCTGTCGTGAA (38) |
| ITGA5 | GCAAGAATCTCAACAACTCGCAA (39) | GCCAGTCGCTCATCGGAAATA (40) |
| ITGA6 | AAGACAGACAGATGATGGCAGA (41) | GGCACTTGATGTTCACACAGTT (42) |
| ITGA9 | AGCCTGTGAACTGCCTCAA (43) | ACATCAGCCGTCAGAACATAAT (44) |
| ITGB1 | CTTCTATTGCTCACCTTGTCCA (45) | AGATAATGTTCCTACCGCTGACTT (46) |
| ITGB4 | CACAACCTCCAGCAGACCAA (47) | CCATCAGCACAGTGTCCACAA (48) |
| ITGB5 | CGGAGAGAGTTCGCCAAGTT (49) | CTGTGCCATTGTAGGATTTGTTGA (50) |
| ITGB6 | CATCAATGAGAAAGACTGTCCAA (51) | CAATAAGAAGAATAGCCAGCGAA (52) |
| Loc518180 | ATACGCACAGCCGCAGA (53) | CACGATGTCACCCACCTCTAA (54) |
| MYH1 | TGAGGAAGCGGAGGAACAAT (55) | TGGGACTCGGCAATGTCA (56) |
| MYH2 | CAATGACCTGACAACCCAGA (57) | CCTTGACAACTGAGACACCAGA (58) |
| NRAS | GCTAATCCAGAACCACTTTGTAGAT (59) | GCCTTCGCCTGTCCTCAT (60) |
| PRDX3 | AAGTTGTGGCAGTGTCAGTGGA (61) | CGTAGTCTCGGGAAATCTGTTTG (62) |
| RPL19 | ACCCCAATGAGACCAATGAA (63) | GCAGTACCCTTTCGCTTACCTAT (64) |
| RPL5 | GACTGGAGATGAATACAATGTGGAA (65) | CGTTTGGTACTGTGAGGGATAGA (66) |
| RPS20 | ACCAGCCGCAACGTGAA (67) | ACCAGCCGCAACGTGAA (68) |
| SDHA | GCAGAACCTGATGCTTTGTG (69) | CGTAGGAGAGCGTGTGCTT (70) |
| TLN1 | AAATGCCAAGAACGGAAACTT (71) | GTCAGACACGCCAACCAGATA (72) |
| TLN2 | GCTGGGACATAAGGTGACACA (73) | CTGCGAGCGTCTTGGTCT (74) |
| UFM1 | AAATAGCCTTCAGGG AGAAAGTGTAA (75) | AATGCTTCCTTAAATGTTCGGTCTTC (76) |
| YWHAZ | GCATCCCACAGACTATTTCC (77) | GCAAAGACAATGACAGACCA (78) |

IDs are based on official gene symbol as provided by NCBI

For all quantitative RT-PCR, the 7900HT Fast Real-Time PCR System (Applied Biosystems, California) was used in the 9600 Fast Emulation mode. This consisted of a 10 min 95° C. melting phase, 40 cycles of 95° C. for 15 s, 60° C. for 1 min, followed by a 95° C. dissociation phase. All reactions were performed in a 20 μL reaction consisting of 6.8 μL RNA-grade water, 10 μL SYBR Green Master Mix, 0.6 μL of each primer, and 2.0 μL of cDNA sample.

SDS (v.2.4) software from Applied Biosystems (Carlsbad, Calif.) was used to quantify qPCR results. A threshold value of 0.20 was used to determine Ct value. Relative expression was calculated using the method described by Livak and Schmittgen (2001). In summary, raw Ct values were normalized to the geometric mean of RPL19, RPL5, and RPS20 based on internal expression stability between groups. The normalized value was subtracted from the raw Ct for each sample (ΔCT). From the ΔCT the average value of the high group was subtracted (ΔΔCT). The ΔΔCT was linearized by taking the inverse negative log-base 2 (RQ). The high group will thus always have a median expression value of 1.0. It should be noted that these are arbitrary units (ΔU) and that no direct comparison between different genes in total expression levels can be reliably made. All values are relative and applicable directly only as a within-group comparison of relative expression.

Based on these expression and pathway analysis a total of 40 genes were selected for validation by qRT-PCR followup. For all qRT-PCR expression values results are normalized to the high shear force group: so results are shown as the fold ratio of expression of the low-WBSF group relative to the high-WBSF group (Table 8). Because of this, the mean expression for every gene in the high group is 1.0 and is not listed. For comparison, the NES group was analyzed in the same manner.

TABLE 8
mRNA relative expression levels for the ES and NES groups

| | ES | | NES | |
|---|---|---|---|---|
| | mRNA Expression | | mRNA Expression | |
| Gene | n = 12 | P-value | n = 12 | P-value |
| 18S | 1.0 ± 0.4 | 0.595 | 0.9 ± 0.1 | 0.645 |
| ACTA1 | 1.1 ± 0.3 | 0.247 | 1.0 ± 0.1 | 0.888 |
| ACTN1 | 0.9 ± 1.5 | 0.831 | 0.8 ± 0.4 | 0.509 |
| ACTN2 | 1.0 ± 0.2 | 0.897 | 0.9 ± 0.0 | 0.483 |
| ACTN3 | 3.8 ± 22.3 | 0.354 | 1.1 ± 0.2 | 0.836 |
| ACTN4 | 1.4 ± 0.3 | 0.003 | 1.1 ± 0.1 | 0.598 |
| B2M | 1.3 ± 0.3 | 0.019 | 1.2 ± 0.1 | 0.154 |
| C4A | 1.3 ± 0.4 | 0.073 | 1.1 ± 0.1 | 0.788 |
| CAPN1 | 1.2 ± 0.3 | 0.051 | 1.0 ± 0.1 | 0.823 |
| CAPN2 | 1.0 ± 0.4 | 0.937 | 1.0 ± 0.1 | 0.931 |
| CAST | 0.7 ± 0.5 | 0.379 | 0.6 ± 0.1 | 0.050 |
| COL1A2 | 1.6 ± 1.0 | 0.087 | 0.8 ± 0.3 | 0.501 |
| COL6A1 | 1.4 ± 0.5 | 0.037 | 1.2 ± 0.4 | 0.588 |
| FLNA | 0.8 ± 2.7 | 0.619 | 0.6 ± 0.9 | 0.282 |
| FLNB | 1.5 ± 0.4 | 0.005 | 1.1 ± 0.1 | 0.681 |
| FLNC | 1.6 ± 1.4 | 0.191 | 1.9 ± 0.5 | 0.103 |
| IPO7 | 1.2 ± 0.3 | 0.140 | 1.1 ± 0.1 | 0.326 |
| ITGA5 | 2.1 ± 0.7 | 0.003 | 1.7 ± 0.5 | 0.104 |
| ITGA6 | 1.6 ± 0.4 | 0.006 | 1.0 ± 0.2 | 0.618 |
| ITGA9 | 1.5 ± 0.3 | <0.001 | 1.1 ± 0.1 | 0.529 |
| ITGB1 | 1.1 ± 0.9 | 0.669 | 1.5 ± 0.3 | 0.084 |
| ITGB4 | 1.7 ± 0.7 | 0.030 | 1.1 ± 0.2 | 0.774 |
| ITGB5 | 1.5 ± 0.7 | 0.113 | 1.1 ± 0.3 | 0.686 |
| ITGB6 | 1.3 ± 0.5 | 0.278 | 1.2 ± 0.2 | 0.223 |
| LOC5181 | 1.0 ± 0.3 | 0.877 | 1.1 ± 0.1 | 0.382 |
| MYH1 | 153.0 ± 781.7 | 0.326 | 1.3 ± 0.2 | 0.529 |
| MYH2 | 2.1 ± 3.2 | 0.172 | 1.7 ± 0.3 | 0.222 |
| NRAS | 0.9 ± 2.0 | 0.896 | 0.7 ± 0.5 | 0.342 |
| PRDX3 | 1.1 ± 0.3 | 0.321 | 1.0 ± 0.1 | 0.918 |
| RPL19 | 1.0 ± 0.1 | 0.229 | 1.0 ± 0.0 | 0.316 |
| RPL5 | 1.0 ± 0.2 | 0.802 | 1.0 ± 0.0 | 0.538 |
| RPS20 | 1.0 ± 0.2 | 0.877 | 1.1 ± 0.0 | 0.134 |
| SHDA | 1.1 ± 0.4 | 0.718 | 1.1 ± 0.1 | 0.619 |
| TLN1 | 1.4 ± 3.0 | 0.537 | 0.9 ± 0.9 | 0.880 |
| TLN2 | 1.2 ± 0.3 | 0.086 | 1.1 ± 0.1 | 0.593 |
| UFM1 | 1.3 ± 0.3 | 0.039 | 1.1 ± 0.1 | 0.368 |
| YWAHZ | 1.3 ± 0.2 | <0.001 | 0.9 ± 0.1 | 0.553 |

Expression values were linearized then normalized to the geometric mean of RPL5, RPL19, and RPS20. Expression is presented as expression fold ratios of the low-WBSF group relative to the high-WBSF group: for example ITGA5 is expressed 2.1 times higher in the low group than the high group among the population selected for extremes of ES residuals.

Several genes were considered for possible use as reference genes for normalization. Myosin heavy chain variant I (MYH1) expression was found to be so variable as to be completely unreliable as a reference gene. Two genes presented as potential reference genes in other studies were significantly different between ES groups: B2M (Perez et al., 2008) and YWAHZ (Goossens et al., 2005). Here, the reference genes used were RPL5, RPL19, and RPS20. All values given for qRT-PCR here were normalized to the geometric mean expression of these 3 genes.

All alpha-integrins assayed were significantly upregulated in the low ES group relative to the high. Beta-integrins were upregulated in the low ES group as well although only ITGB4 was significant (P=0.03). One collagen, COL6A1, was significantly upregulated in the low ES group as well. COL1A2 was also upregulated in the low ES group, but it fell short of significance. This finding is interesting as all previous studies found that increased collagen was associated with increased toughness.

With the exception of calpastatin (CAST) no genes in this set were differentially expressed to a significant level between groups when based on NES residuals. Calpastatin was expressed at a lower level in those carcasses that responded most favorably in terms of WBSF to aging in the absence of electrical stimulation. Based on previous studies, this result is not surprising (Koohmaraie et al., 1988; Taylor et al., 1995b; Delgado et al., 2001; Geesink et al., 2006). Calpastatin expression was not significant in relation to WBSF following ES treatment, although μ-calpain (CAPN1) came close to significance (P=0.051) and was upregulated in the low ES group (1.22), as might be expected.

Quantitative RT-PCR results for the 24 samples assayed for gene expression showed significant differences in expression in components of these pathways between ES tenderness groups. ACTN4, COL6A1, FLNB, ITGA5, ITGA6, ITGA9, and ITGB4 were all significantly upregulated in the low WBSF-group (1.4, 1.5, 1.4, 2.1, 1.6, 1.5, and 1.7 fold, respectively) compared with the high-WBSF group. With the exception of CAST (1.6 fold higher in the tough group), no genes were expressed differently at a significant level between groups selected for NES tenderness. Higher expression of CAST leading to increased toughness by suppression of calpain-induced proteolysis is well supported by the literature (Whipple et al., 1990a; Delgado et al., 2001; Hope-Jones et al., 2010). What was interesting is that CAST expression failed to even approach significance in animals selected for extremes of tenderness following ES. This indicates that ES interacts with, or overshadows, the effects of calpain-induced proteolysis in tenderization.

### Example 8

### Haplotype Analysis

Haplotype analysis was performed as follows. All individuals in the first three generations of the McGregor Genomics population were genotyped with the Illumina BovSNP50 v1 assay. Genotypes were filtered to remove animals with poor completion rate, SNPs with low minor allele frequency, poor completion rate, and those SNPs that deviated from Hardy-Weinberg equilibrium. To determine whether breed and parent of origin played a role in the efficiency phenotype, SNP genotypes spanning 1 Mb intervals flanking several genes of interest based on expression analyses were extracted using PLINK v1.07 (Purcell et al., 2007) and formatted for phase v2.1.1 software (Stephens et al., 2001). Haplotypes were established using 100 iterations of phase v2.1.1, with a thinning interval of 2 and a burn-in of 10. Resultant phased haplotypes were ordered by generation, and breed (Nellore or Angus) and parent of origin were manually tracked through the pedigree to assign the source of each haplotype block (hapblock) in the F2 generation.

Genes selected for haplotype analysis were those that either showed significant differences in gene expression as assessed by qPCR, were found in pathways of interest, were shown to be of interest by a literature search, or some combination of the three. These genes can also be classified in to 3 main functional categories: the calpain system, muscle structural proteins, and components of the ECM.

A list of these genes along with chromosome location is given in Table 9. The total number of SNPs present within the defined region is listed in the “SNP in hapblock” column.

TABLE 9
Location of genes used in the construction of hapblocks

| | | | | | Infor- |
|---|---|---|---|---|---|
| | | | | | mative |
| | | Chro- | | | SNP in |
| Category | Gene | mosome | Coordinates | | hapblock |
| Calpain | CAPN1 | 29 | 44063463 | 44113492 | 24 |
| System | CAPN2 | 16 | 27781671 | 27840011 | 24 |
| | CAPN3 | 10 | 37829007 | 37885645 | 24 |
| | CAST | 7 | 98444979 | 98581253 | 18 |
| Muscle | ACTN2 | 28 | 9403203 | 9450920 | 16 |
| Structure | ACTN3 | 29 | 45230630 | 45242406 | 16 |
| | MYH1/2 | 19 | 30110728 | 30165109 | 18 |
| ECM/ | ITGA5 | 5 | 25778012 | 25799053 | 14 |
| Focal | ITGA6 | 2 | −24230649 | −24118992 | 18 |
| Adhesion | ITGA9 | 22 | 10892055 | 11361218 | 23 |
| | ITGB1 | 13 | 20248978 | 20290982 | 11 |
| | ITGB4 | 19 | −56511970 | −56467165 | 20 |
| | ITGB5 | 1 | −69914350 | −69787160 | 15 |
| | ITGB6 | 2 | 36242459 | 36362623 | 16 |
| | FN1 | 2 | 103881402 | 103950562 | 12 |

Coordinates are based on the UMD 3.1 bovine genome assembly (Bos_{—}taurus_UMD_{—}3.1, GenBank assembly ID GCA_{—}000003055.3).

Hapblock analyses was performed on the larger population (n=196). A total of 15 unique hapblocks were examined for breed and parent of origin effects. Based on the literature (Koohmaraie, 1994; Taylor et al., 1995a; Casas et al., 2006; Geesink et al., 2006) the calpain system was selected and consisted of μ-calpain (CAPN1), m-calpain (CAPN2), calpain p94 (CAPN3), and the endogenous inhibiting element calpastatin (CAST). These were not significant in either the pathway or expression analysis in this study with the exception of CAST mRNA expression in NES. They are heavily cited as playing a role in postmortem beef tenderization in the literature. For this reason they were selected for haplotype analysis.

Muscle structural genes studied were α-actinin 2 and α-actinin 3 (ACTN2 and ACTN3), and the myosin heavy chain components MYH1 and MYH2. The actinin genes were significant in pathway analysis. The myosin heavy chain structures were a downstream component of the focal adhesion and ECM regulation pathways although those 2 were not found to be significant in terms of expression analysis, possibly due to variability in expression. MYH1 and MYH2 were located in close proximity to each other and are found within the same 1 megabase region so only one haplotype analysis was performed that encompassed both genes and surrounding elements (labeled here as MYH1/2).

Finally, components of the ECM and focal adhesions were analyzed. The majority of these were integrins (ITGA5, ITGA6, ITGA9, ITGB1, ITGB4, ITGB5, and ITGB6). Fibronectin 1 (FN1) was also included although it was not assayed for expression. Fibronectin is a signaling and structural component that interacts with various integrin dimers in the living tissue. FN1 was not significant in the pathway or expression analysis. However, ITGA5 and ITGB1 form a complex that in muscle is a fibronectin receptor (Mayer, 2003). Among the calpains only CAPN3 was linked to a difference in tenderness (P=0.04). The paternally inherited Angus CAPN3 hapblock was linked to an improvement in tenderness compared to the Nellore hapblock (WBSF ES residuals 0.06 and −0.08, respectively). The paternally inherited CAST hapblock approached significance (P=0.08), with the Angus hapblock having an improved residual tenderness when compared to the paternally inherited Nellore hapblock (0.12 and −0.12, respectively) (Table 10).

TABLE 10
WBSF residuals grouped by hapblocks in the Calpain system

| | | | | | | Paternal | | | Maternal | | |
|---|---|---|---|---|---|---|---|---|---|---|---|
| | | Hapblock | | | | | | P- | | | P- |
| | | NN | NA | AN | AA | NN/NA | AN/AA | value | NN/AN | NA/AA | value |
| CAST | n | 51 | 49 | 51 | 45 | 100 | 96 | | 102 | 94 | |
| | ES | 0.03 ± 0.06 | 0.03 ± 0.09 | −0.04 ± 0.06 | −0.06 ± 0.07 | 0.03 ± 0.05 | −0.05 ± 0.05 | 0.25 | 0.00 ± 0.04 | −0.01 ± 0.06 | 0.89 |
| | NES | 0.12 ± 0.14 | 0.11 ± 0.15 | −0.02 ± 0.12 | −0.24 ± 0.13 | 0.12 ± 0.10 | −0.12 ± 0.09 | 0.08 | 0.05 ± 0.09 | −0.06 ± 0.10 | 0.42 |
| CAPN1 | n | 28 | 45 | 39 | 84 | 73 | 123 | | 67 | 129 | |
| | ES | 0.00 ± 0.07 | 0.03 ± 0.08 | −0.04 ± 0.08 | −0.01 ± 0.05 | 0.02 ± 0.05 | −0.02 ± 0.05 | 0.60 | −0.02 ± 0.06 | 0.00 ± 0.04 | 0.70 |
| | NES | 0.26 ± 0.21 | −0.05 ± 0.15 | −0.13 ± 0.14 | 0.00 ± 0.10 | 0.07 ± 0.12 | −0.04 ± 0.08 | 0.42 | 0.04 ± 0.12 | −0.02 ± 0.08 | 0.70 |
| CAPN2 | n | 44 | 63 | 45 | 44 | 107 | 89 | | 89 | 107 | |
| | ES | 0.11 ± 0.08 | 0.02 ± 0.06 | −0.07 ± 0.07 | −0.09 ± 0.07 | 0.06 ± 0.05 | −0.08 ± 0.05 | 0.06 | 0.02 ± 0.05 | −0.03 ± 0.04 | 0.51 |
| | NES | 0.28 ± 0.16 | −0.03 ± 0.12 | −0.10 ± 0.13 | −0.13 ± 0.12 | 0.10 ± 0.10 | −0.12 ± 0.09 | 0.12 | 0.09 ± 0.11 | −0.07 ± 0.09 | 0.23 |
| CAPN3 | n | 42 | 63 | 44 | 47 | 105 | 91 | | 86 | 110 | |
| | ES | 0.13 ± 0.08 | 0.01 ± 0.06 | −0.07 ± 0.08 | −0.09 ± 0.07 | 0.06 ± 0.05 | −0.08 ± 0.05 | 0.04 | 0.03 ± 0.06 | −0.03 ± 0.04 | 0.38 |
| | NES | 0.22 ± 0.17 | −0.01 ± 0.12 | −0.10 ± 0.14 | −0.10 ± 0.12 | 0.09 ± 0.10 | −0.10 ± 0.09 | 0.17 | 0.06 ± 0.11 | −0.05 ± 0.09 | 0.45 |

ES and NES values are given as RFI_{(NRC)} residuals, kg.

The different calpain hapblocks could not be linked significantly to differences in expression, as measured by qRT-PCR, of CAPN1, CAPN2, CAPN3, and CAST in the population assayed.

Two hapblocks from the focal adhesion pathway were significant. The FN1 Angus hapblock was linked to an improvement in ES and NES tenderness compared to the Nellore hapblock when inherited paternally (P<0.01 and P=0.02, respectively). The ITGA6 Nellore hapblock was linked to an improvement in ES tenderness when inherited maternally (P=0.03). Additionally the ITGB6 hapblock came close to significance (P=0.08) with the Nellore being associated with an improved ES tenderness relative to the Angus hapblock. The ITGA9 hapblock also approached significance (P=0.09) with the Angus hapblock being associated with an improved ES tenderness relative to the Nellore hapblock. Other than the FN1 hapblock no genes assayed in the focal adhesion pathway had any significant effect on NES residual tenderness (Table 11).

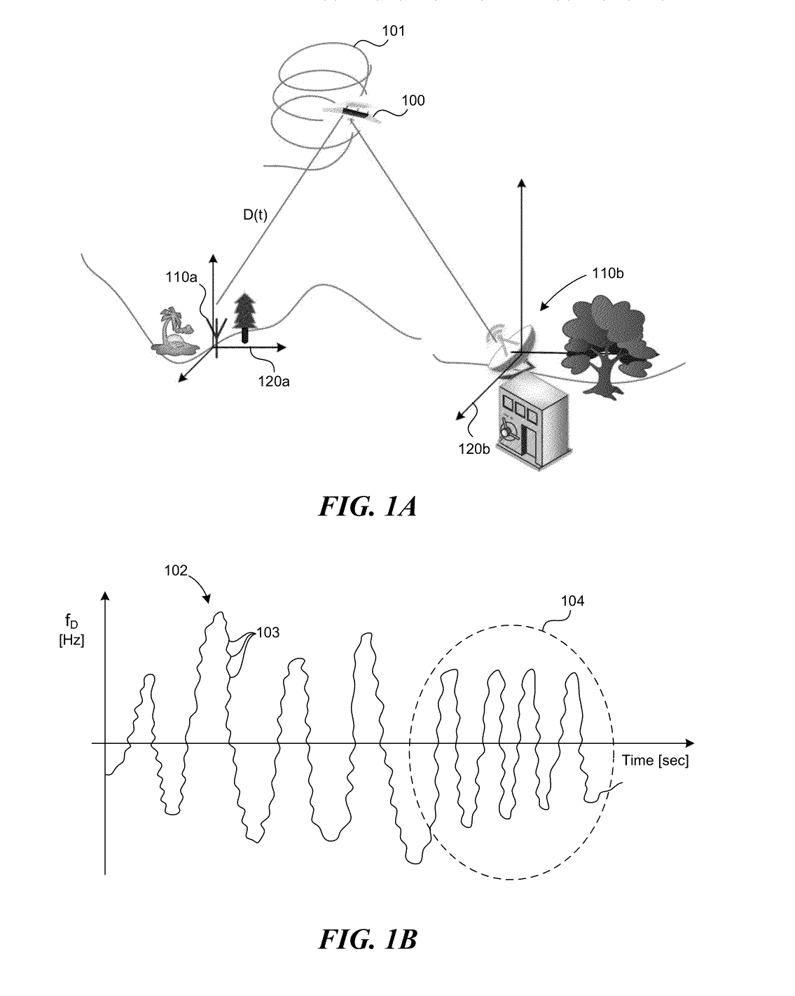

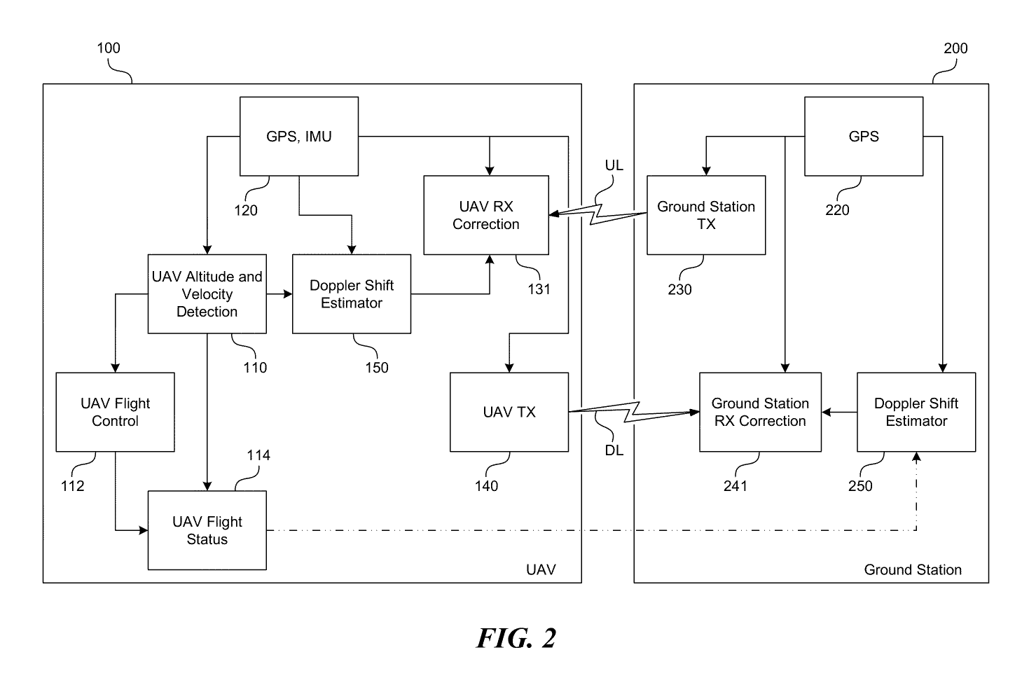

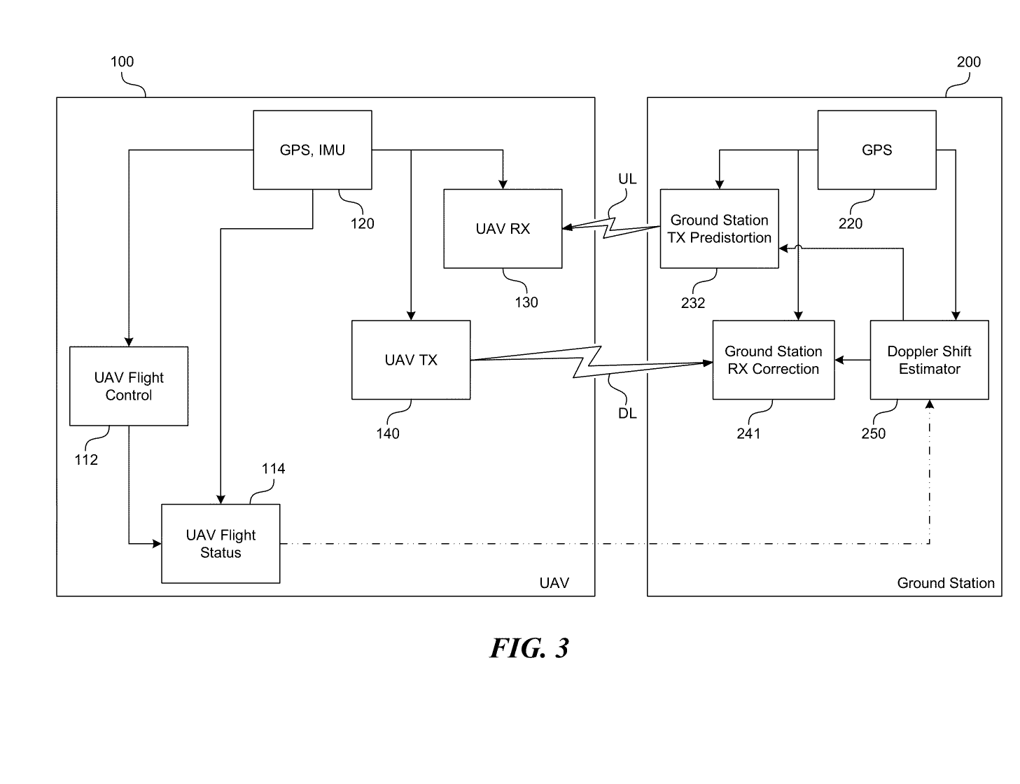

As a measure of ES tenderness, hapblocks encompassing components of the focal adhesion pathway were found to be more useful than hapblocks encompassing CAPN1, CAPN2, and CAST. The ITGA6 and FN1 genes both showed significant breed of origin effects for ES tenderness (FIGS. 1 and 2). The Nellore ITGA6 was associated with a reduction in ES residual tenderness by 0.15 kg when inherited maternally. The Angus FN1 hapblock was associated with a reduction in ES residual tenderness by 0.23 kg and a reduction of NES residual tenderness by 0.33 kg when inherited paternally (P<0.01 and P=0.02, respectively). The ITGB6 hapblock had a similar effect (reduction of ES residual tenderness by 0.12 kg for the maternal Nellore hapblock) to the ITGA6 hapblock although it fell short of significance (P=0.08) (FIG. 3). ITGB6 mRNA expression levels were not significantly different between groups (P=0.278). SNPs corresponding to the hapblocks for ITGA6, FN1, and ITGB6 are provided in Tables 12-14, respectively.

TABLE 11
WBSF residuals grouped by hapblocks in the focal adhesion and ECM pathways

| | | Hapblock | | | | Paternal |
|---|---|---|---|---|---|---|
| | | NN | NA | AN | AA | NN/NA |
| ITGB1 | N | 62 | 60 | 39 | 35 | 122 |
| | ES | 0.07 ± 0.06 | 0.00 ± 0.06 | −0.07 ± 0.08 | −0.07 ± 0.08 | 0.03 ± 0.04 |
| | NES | −0.01 ± 0.12 | 0.07 ± 0.12 | −0.21 ± 0.16 | 0.14 ± 0.16 | 0.03 ± 0.08 |
| ITGB4 | N | 37 | 50 | 55 | 54 | 87 |
| | ES | 0.00 ± 0.09 | 0.01 ± 0.07 | −0.02 ± 0.06 | −0.01 ± 0.07 | 0.00 ± 0.05 |
| | NES | 0.05 ± 0.15 | 0.11 ± 0.13 | −0.07 ± 0.14 | −0.06 ± 0.12 | 0.08 ± 0.10 |
| ITGB5 | N | 37 | 52 | 57 | 50 | 89 |
| | ES | −0.11 ± 0.06 | 0.00 ± 0.06 | 0.02 ± 0.07 | 0.03 ± 0.08 | −0.04 ± 0.04 |
| | NES | 0.03 ± 0.16 | 0.00 ± 0.13 | −0.05 ± 0.14 | 0.03 ± 0.12 | 0.01 ± 0.10 |
| ITGB6 | N | 53 | 48 | 40 | 55 | 101 |
| | ES | −0.10 ± 0.06 | −0.02 ± 0.07 | 0.05 ± 0.09 | 0.06 ± 0.07 | −0.06 ± 0.04 |
| | NES | −0.01 ± 0.13 | 0.03 ± 0.15 | −0.05 ± 0.15 | 0.02 ± 0.13 | 0.01 ± 0.10 |
| ITGA5 | N | 54 | 41 | 59 | 42 | 95 |
| | ES | 0.01 ± 0.08 | −0.02 ± 0.06 | 0.00 ± 0.07 | −0.02 ± 0.05 | 0.00 ± 0.05 |
| | NES | −0.07 ± 0.13 | −0.13 ± 0.15 | 0.07 ± 0.12 | 0.13 ± 0.14 | −0.10 ± 0.10 |
| ITGA6 | N | 42 | 56 | 39 | 59 | 98 |
| | ES | −0.18 ± 0.07 | 0.04 ± 0.06 | −0.01 ± 0.08 | 0.07 ± 0.07 | −0.05 ± 0.05 |
| | NES | −0.14 ± 0.13 | 0.00 ± 0.14 | 0.10 ± 0.16 | 0.04 ± 0.11 | −0.06 ± 0.10 |
| ITGA9 | N | 57 | 40 | 44 | 55 | 97 |
| | ES | 0.08 ± 0.07 | 0.02 ± 0.09 | −0.03 ± 0.07 | −0.09 ± 0.06 | 0.05 ± 0.05 |
| | NES | 0.03 ± 0.11 | −0.03 ± 0.15 | 0.07 ± 0.16 | −0.06 ± 0.13 | 0.00 ± 0.09 |
| FN1 | N | 68 | 62 | 31 | 35 | 130 |
| | ES | 0.10 ± 0.06 | 0.05 ± 0.06 | −0.09 ± 0.07 | −0.22 ± 0.08 | 0.07 ± 0.04 |
| | NES | 0.04 ± 0.12 | 0.19 ± 0.12 | −0.18 ± 0.17 | −0.25 ± 0.15 | 0.11 ± 0.08 |

| | | Paternal | | Maternal | | |
|---|---|---|---|---|---|---|
| | | | P- | | | P- |
| | | AN/AA | value | NN/AN | NA/AA | value |
| ITGB1 | N | 74 | | 101 | 95 | |
| | ES | −0.07 ± 0.06 | 0.15 | 0.01 ± 0.05 | −0.02 ± 0.05 | 0.60 |
| | NES | −0.04 ± 0.11 | 0.62 | −0.09 ± 0.10 | 0.09 ± 0.10 | 0.18 |
| ITGB4 | N | 109 | | 92 | 104 | |
| | ES | −0.01 ± 0.05 | 0.85 | −0.01 ± 0.05 | 0.00 ± 0.05 | 0.88 |
| | NES | −0.07 ± 0.09 | 0.27 | −0.02 ± 0.10 | 0.02 ± 0.09 | 0.73 |
| ITGB5 | N | 107 | | 94 | 102 | |
| | ES | 0.03 ± 0.05 | 0.32 | −0.03 ± 0.05 | 0.02 ± 0.05 | 0.53 |
| | NES | −0.01 ± 0.09 | 0.85 | −0.02 ± 0.11 | 0.02 ± 0.09 | 0.80 |
| ITGB6 | N | 95 | | 93 | 103 | |
| | ES | 0.06 ± 0.05 | 0.08 | −0.04 ± 0.05 | 0.02 ± 0.05 | 0.40 |
| | NES | −0.01 ± 0.10 | 0.92 | −0.03 ± 0.10 | 0.02 ± 0.10 | 0.70 |
| ITGA5 | N | 101 | | 113 | 83 | |
| | ES | −0.01 ± 0.05 | 0.95 | 0.01 ± 0.05 | −0.02 ± 0.04 | 0.69 |
| | NES | 0.09 ± 0.09 | 0.15 | 0.00 ± 0.09 | 0.00 ± 0.10 | 0.97 |
| ITGA6 | N | 98 | | 81 | 115 | |
| | ES | 0.04 ± 0.05 | 0.19 | −0.09 ± 0.05 | 0.06 ± 0.04 | 0.03 |
| | NES | 0.06 ± 0.09 | 0.37 | −0.02 ± 0.10 | 0.02 ± 0.09 | 0.76 |
| ITGA9 | N | 99 | | 101 | 95 | |
| | ES | −0.06 ± 0.05 | 0.09 | 0.03 ± 0.05 | −0.04 ± 0.05 | 0.29 |
| | NES | 0.00 ± 0.10 | 0.95 | 0.05 ± 0.10 | −0.05 ± 0.10 | 0.48 |
| FN1 | N | 66 | | 99 | 97 | |
| | ES | −0.16 ± 0.05 | <0.01 | 0.04 ± 0.05 | −0.05 ± 0.05 | 0.21 |
| | NES | −0.22 ± 0.11 | 0.02 | −0.03 ± 0.10 | 0.03 ± 0.10 | 0.68 |

ES and NES values are given as RFI_{(NRC)} residuals, kg.

TABLE 12
SNPs corresponding to ITGA6 hapblock on bovine chromosome 2

| SNP ID | Position |
|---|---|
| BTB-00085877 | 23628756 |
| ARS-BFGL-NGS-14141 | 23654385 |
| Hapmap59009-rs29027446 | 23686403 |
| ARS-BFGL-NGS-103110 | 23715994 |
| Hapmap30793-BTA-133756 | 23757340 |
| BTB-01693574 | 23792090 |
| ARS-BFGL-NGS-81142 | 23824758 |
| ARS-BFGL-NGS-29277 | 23861940 |
| ARS-BFGL-NGS-117584 | 23904611 |
| Hapmap40436-BTA-46804 | 23936626 |
| Hapmap58163-rs29021257 | 24039833 |
| ARS-BFGL-NGS-29474* | 24147156 |
| BTB-00086875* | 24168854 |
| BTA-46847-no-rs* | 24192141 |
| BTB-00086968* | 24221002 |
| BTA-46852-no-rs | 24250783 |
| BTB-00087067 | 24285533 |
| ARS-BFGL-NGS-10872 | 24325003 |
| ARS-BFGL-NGS-99222 | 24362479 |
| ARS-BFGL-NGS-14875 | 24403108 |
| ARS-BFGL-NGS-4499 | 24441912 |
| Hapmap52128-rs29015710 | 24472770 |
| Hapmap57629-rs29027188 | 24487457 |
| Hapmap59917-rs29012418 | 24519348 |
| BTB-00087562 | 24543348 |
| ARS-BFGL-NGS-43595 | 24567533 |
| BTB-00087717 | 24592039 |
| ARS-BFGL-NGS-1992 | 24616727 |
| BTB-00087742 | 24639094 |

*Indicates SNPs that fall within the ITGA6 gene.

TABLE 13
SNPs corresponding to FN1 hapblock on bovine chromosome 2

| SNP ID | Position |
|---|---|
| ARS-BFGL-NGS-96747 | 103421027 |
| BTA-48534-no-rs | 103470685 |
| Hapmap49191-BTA-48538 | 103491995 |
| ARS-BFGL-NGS-3072 | 103528661 |
| BTA-05667-rs29019825 | 103592247 |
| ARS-BFGL-BAC-5705 | 103707675 |
| ARS-BFGL-BAC-35929 | 103731623 |
| Hapmap26477-BTA-161779 | 103810327 |
| ARS-BFGL-NGS-117549* | 103864593 |
| ARS-BFGL-BAC-35928* | 103890577 |
| UA-IFASA-6155* | 103916046 |
| ARS-BFGL-NGS-4334* | 103947921 |
| BTA-48084-no-rs | 103995601 |
| ARS-BFGL-NGS-23725 | 104026376 |
| Hapmap50004-BTA-48076 | 104083149 |
| Hapmap33475-BTA-134171 | 104123985 |
| ARS-BFGL-BAC-35925 | 104145868 |
| BTA-97778-no-rs | 104256806 |
| ARS-BFGL-NGS-30643 | 104297699 |
| Hapmap43422-BTA-97780 | 104320886 |
| ARS-BFGL-NGS-75769 | 104349734 |

*Indicates SNPs that fall within or flank the FN1 gene.

TABLE 14
SNPs corresponding to ITGB6 hapblock on bovine chromosome 2

| SNP ID | Position |
|---|---|
| BTB-01246228 | 35727838 |
| Hapmap24346-BTA-158052 | 35761857 |
| BTB-00295753 | 35827910 |
| BTB-00295725 | 35852731 |
| ARS-BFGL-NGS-8633 | 35898373 |
| ARS-BFGL-BAC-3633 | 35936557 |
| Hapmap50820-BTA-91209 | 35993314 |
| ARS-BFGL-NGS-32446 | 36077493 |
| Hapmap26705-BTA-149119 | 36178912 |
| ARS-BFGL-NGS-85296 | 36206558 |
| ARS-BFGL-NGS-110726* | 36228793 |
| Hapmap25251-BTA-133896* | 36252633 |
| ARS-BFGL-NGS-15574* | 36329629 |
| ARS-BFGL-NGS-117164* | 36393032 |
| Hapmap46826-BTA-47345 | 36441803 |
| Hapmap27039-BTA-22947 | 36570379 |
| Hapmap39151-BTA-22132 | 36603752 |
| ARS-BFGL-NGS-102089 | 36655521 |
| Hapmap33597-BTA-154241 | 36686850 |
| Hapmap42250-BTA-55589 | 36850914 |

*Indicates SNPs that fall within or flank the ITGB6 gene.

No integrin hapblocks used here were associated with any differences in integrin mRNA expression. Nellore hapblocks were associated with a general increase in integrin expression however this trend was not universal or significant.

Of the remaining muscle component hapblocks only paternally inherited ACTN3 had any significant impact on tenderness (Table 15). A paternally inherited Angus ACTN3 hapblock is associated with a lower ES (P=0.02). No other muscle component hapblocks approached significance for either ES or NES residual tenderness. No muscle component hapblocks could be linked significantly to any differences in mRNA expression, as measured by qRT-PCR, for the muscle component genes assayed.

ACTN3 is a muscle specific structural protein found only in fast twitch muscle fibers. Fiber type differences have been associated with differences in eating quality in beef previously (Calkins et al., 1981; Chikuni et al., 2010) as well as proteolytic capacity (Burniston et al., 2005a; Burniston et al., 2005b; Kocturk et al., 2008; McMillan and Quadrilatero, 2011). Haplotype differences in the ACTN3 gene only affected tenderness when inherited paternally.

TABLE 15
WBSF residuals grouped by hapblocks in the muscle structural component pathways

| | | Hapblock | | | | Paternal |
|---|---|---|---|---|---|---|
| | | NN | NA | AN | AA | NN/NA |
| ACTN2 | n | 50 | 46 | 45 | 55 | 96 |
| | ES | −0.06 ± 0.07 | 0.01 ± 0.05 | 0.07 ± 0.09 | −0.03 ± 0.07 | −0.03 ± 0.04 |
| | NES | 0.02 ± 0.12 | 0.08 ± 0.16 | −0.17 ± 0.11 | 0.05 ± 0.15 | 0.05 ± 0.10 |
| ACTN3 | n | 43 | 62 | 44 | 47 | 105 |
| | ES | 0.13 ± 0.08 | 0.02 ± 0.06 | −0.09 ± 0.07 | −0.09 ± 0.07 | 0.07 ± 0.05 |
| | NES | 0.23 ± 0.17 | −0.04 ± 0.12 | −0.08 ± 0.13 | −0.08 ± 0.12 | 0.07 ± 0.10 |
| MYH1/2 | n | 32 | 48 | 61 | 55 | 80 |
| | ES | 0.05 ± 0.10 | −0.02 ± 0.06 | 0.03 ± 0.07 | −0.07 ± 0.06 | 0.01 ± 0.05 |
| | NES | 0.03 ± 0.15 | −0.15 ± 0.14 | 0.04 ± 0.12 | 0.06 ± 0.14 | −0.07 ± 0.10 |

| | | Paternal | | Maternal | | |
|---|---|---|---|---|---|---|
| | | AN/AA | value | NN/AN | NA/AA | value |
| ACTN2 | n | 100 | | 95 | 101 | |
| | ES | 0.02 ± 0.06 | 0.53 | 0.00 ± 0.05 | −0.01 ± 0.04 | 0.86 |
| | NES | −0.05 ± 0.10 | 0.47 | −0.07 ± 0.08 | 0.06 ± 0.11 | 0.33 |
| ACTN3 | n | 91 | | 87 | 109 | |
| | ES | −0.09 ± 0.05 | 0.02 | 0.02 ± 0.06 | −0.02 ± 0.04 | 0.54 |
| | NES | −0.08 ± 0.09 | 0.26 | 0.07 ± 0.11 | −0.06 ± 0.09 | 0.33 |
| MYH1/2 | n | 116 | | 93 | 103 | |
| | ES | −0.01 ± 0.05 | 0.73 | 0.04 ± 0.06 | −0.04 ± 0.04 | 0.24 |
| | NES | 0.05 ± 0.09 | 0.37 | 0.04 ± 0.09 | −0.03 ± 0.10 | 0.60 |

ES and NES values are given as RFI_{(NRC)} residuals, kg.

### Example 9

### Protein Analysis

From a subset of 8 samples (8208, 8133, 7123, 7115, 8156, 7232, 8157, and 8050) protein was extracted in RIPA buffer from approximately 1 g sample stored at −80° C. The solution used for RIPA was: 73.5 mL H2O, 5.0 mL 3M NaCl, 5.0 mL conc Tris HCl, 10.0 mL, 10% Triton X-100, 0.2 mL 0.5 M EDTA (pH 8.0), 1.0 mL protease inhibitor, 1 mL phosphatase inhibitor cocktail I and 1 mL phosphatase inhibitor cocktail II. Protein samples were quantified using Coomassie Plus Bradford Assay Kit supplied by Pierce Biotechnology (Rockford, Ill.) using serum albumin as a standard. Samples were diluted to a standard concentration based on quantity as determined by quadratic best fit curve of absorbance at 595 nm.

A 5% stacking gel and 10% resolving gel were used for sufficient separation of bands. The stacking gel recipe was 2.7 mL H2O, 0.67 mL 30% acrylamide mix, 0.5 mL 1.0M Tris (pH 6.8), 0.04 mL 10% SDS, 0.04 mL 10% ammonium persulfate, and 0.004 mL TEMED. The resolving gel recipe was 4.0 mL H2O, 3.3 mL 30% acrylamide mix, 2.5 mL 1.5M Tris (pH 8.8), 0.1 mL 10% SDS, 0.1 mL 10% ammonium persulfate, and 0.004 mL TEMED. Gels were run in pairs using a Bio-Rad Mini-PROTEAN Tetra Cell setup according to manufacturer's instructions. A total of 25 μg of sample in 30 μL Laemmli buffer were added to each well. Gel separation and blotting were both performed at 100V for 1 h. Antibodies were diluted according to manufacturer's instructions in a solution of 7.5% dry milk solution in TBST according to lab protocol. Primary antibodies were allowed to bind overnight (12 or more h) with steady agitation at 4° C. Secondary antibodies were allowed to attach for 1 h at room temperature. All secondary antibodies contained horse radish peroxidase and were labeled using the ECL Western Blotting Detection kit provided by GE Healthcare (Waukesha, Wis.). Fluorescence was detected by the Gel Doc XR and ChemiDoc XRS gel documentation system and quantified by Quantity One 1-D Analysis software by Bio-Rad (Hercules, Calif.). The antibodies used are presented in Table 16.

TABLE 16
Antibodies used in western blot analysis

| | | | | Product |
|---|---|---|---|---|
| Antibody | kDa | Target | Company | number |
| ITGB1 | 138 | N20 | Santa Cruz Biotechnology | sc-6622 |
| ITGA6 | 120 | C18 | Santa Cruz Biotechnology | sc-6596 |
| ITGB6 | 97 | N20 | Santa Cruz Biotechnology | sc-6633 |
| m-CAPN | 80 | 80 kDa | Thermo Scientific | MA3-942 |
| u-CAPN | 80 | 80 kDa | Thermo Scientific | MA3-940 |
| CAST | 80 | 80 kDa | Thermo Scientific | MA3-944 |
| anti-goat | | | Santa Cruz Biotechnology | sc-2304 |
| secondary | | | Inc. | |
| anti-mouse | | | Thermo Scientific | SA1-72018 |
| secondary | | | | |

Integrins are compatible with anti-goat antibody and the calpains and calpastatin were compatible with the anti-mouse antibody. All were compatible with use in bovine tissues according to manufacturer's specifications.

To verify that gene expression differences could be tied to protein differences a western blot analyses was performed on a subset (n=8) of the samples used in the qRTPCR study. For the western blot analyses samples 8208, 8133, 7123, and 7115 were selected from the tender group (mean ES WBSF residuals of −0.98) and samples 8156, 7232, 8157, and 8050 were selected from the tough group (mean ES WBSF residuals of 1.43). These 2 groups were significantly different in WBSF ES residuals, but not in WBSF NES residuals (Table 17).

TABLE 17
Mean tenderness values for sample subset used in western blot analyses

| | High | Low | P-value |
|---|---|---|---|
| N | 4 | 4 | |
| ES Residuals, kg | 1.43 | −0.98 | <0.001 |
| NES Residuals, kg | −0.33 | −0.48 | 0.832 |

A total of 8 animals were used for protein quantification based on extremes of ES residuals. The NES residuals for these animals is also listed for comparison purposes. Mean values are listed.

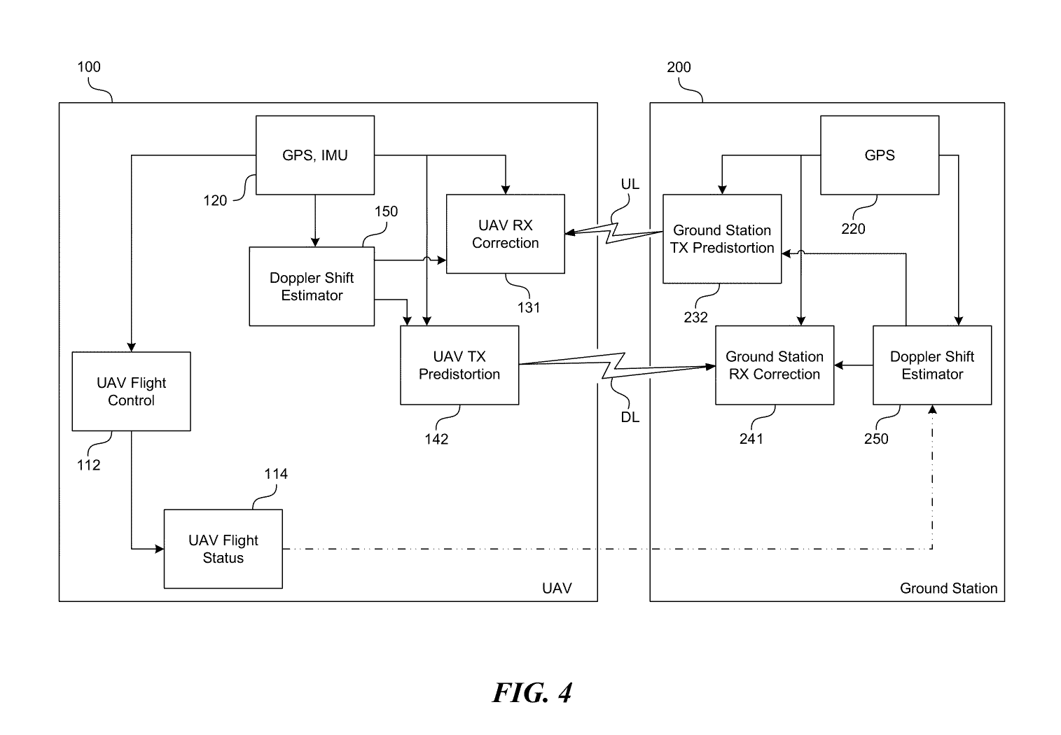

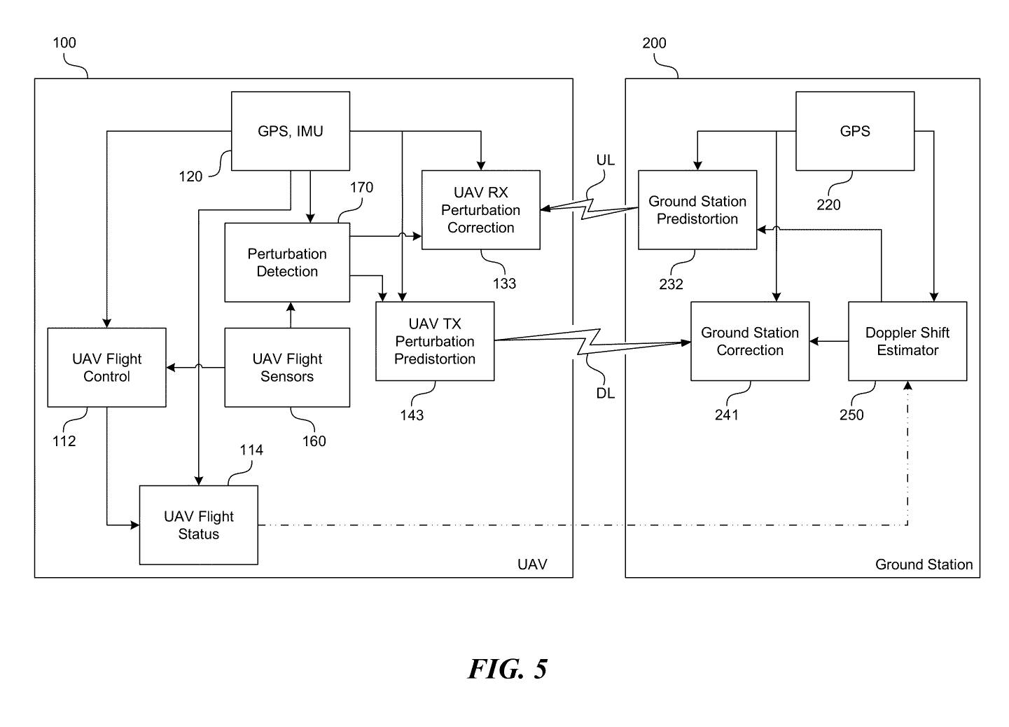

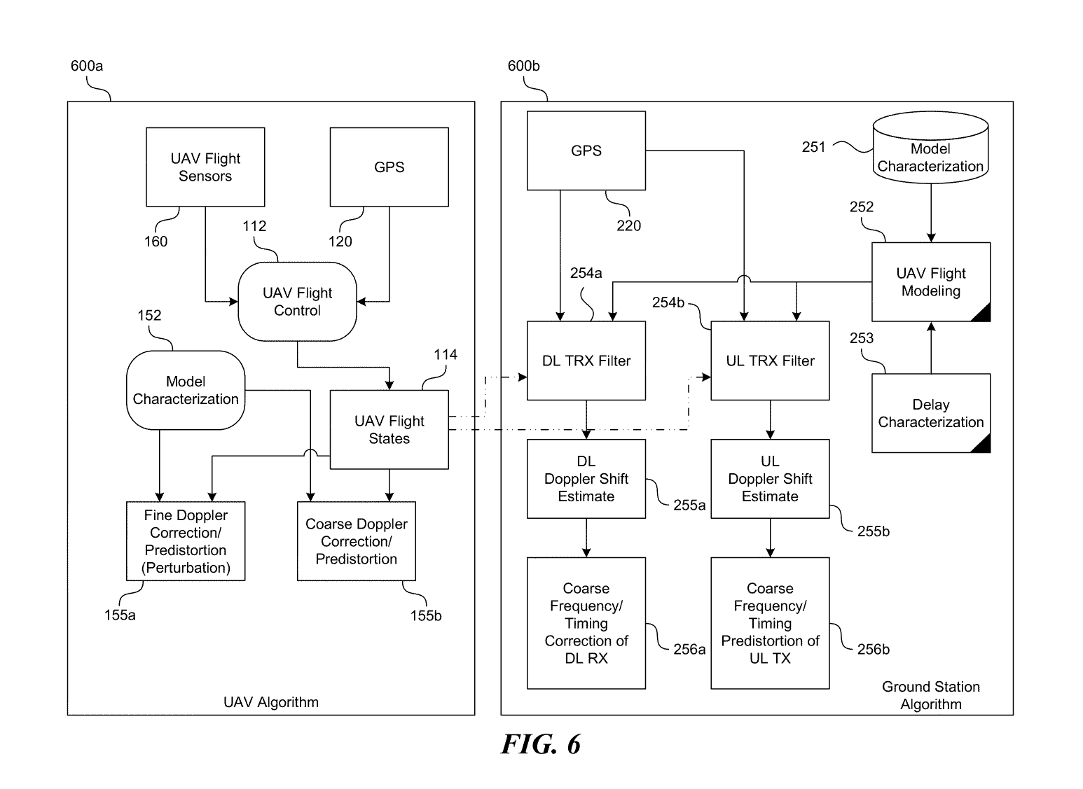

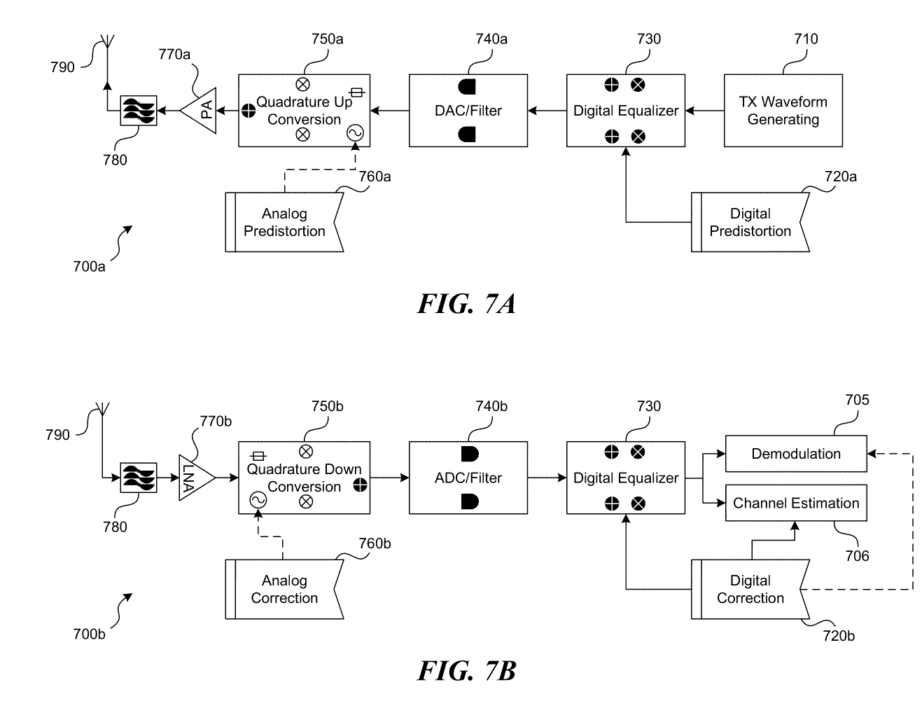

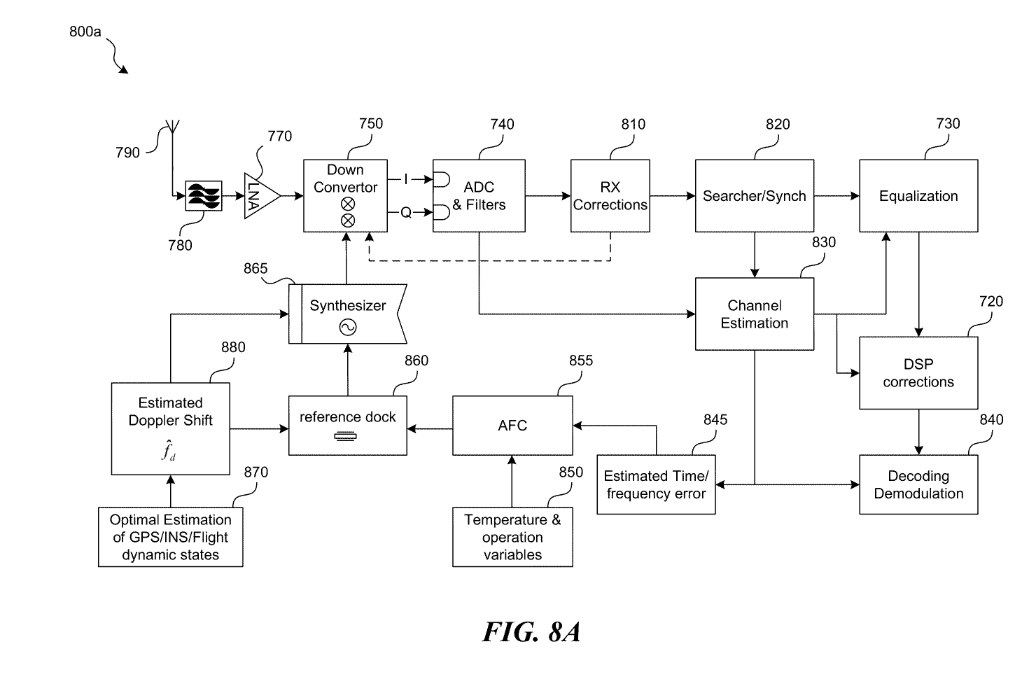

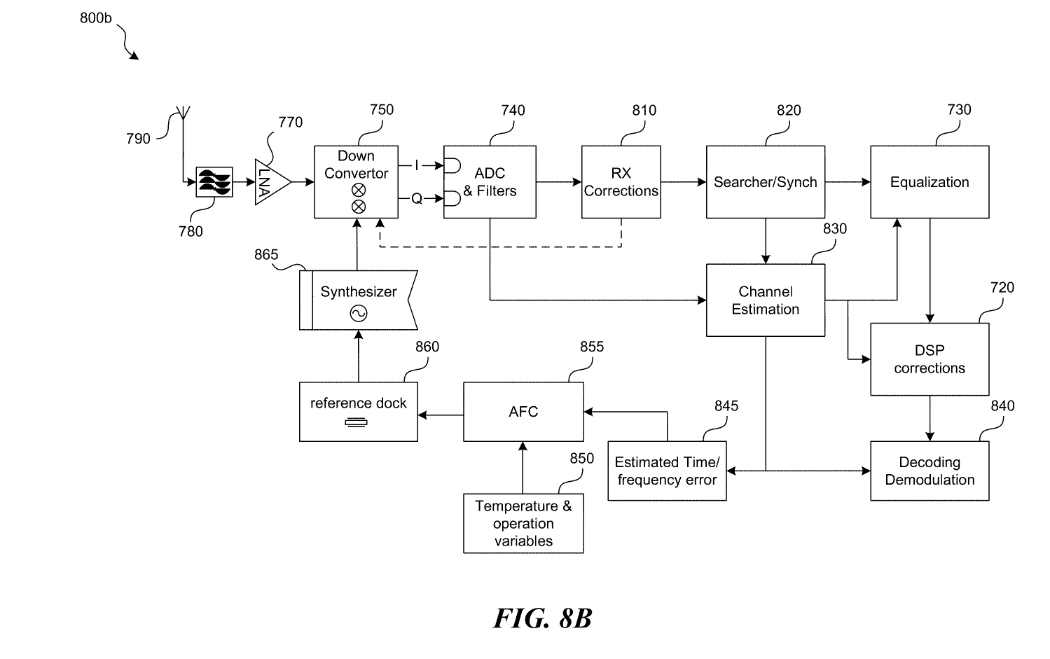

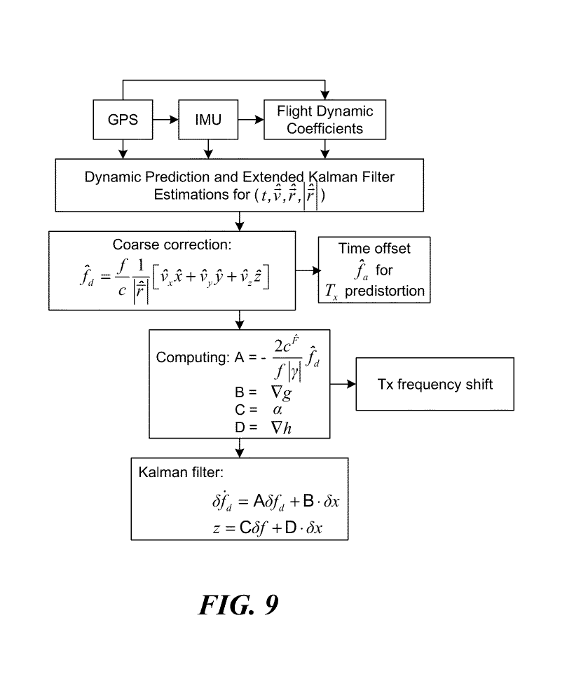

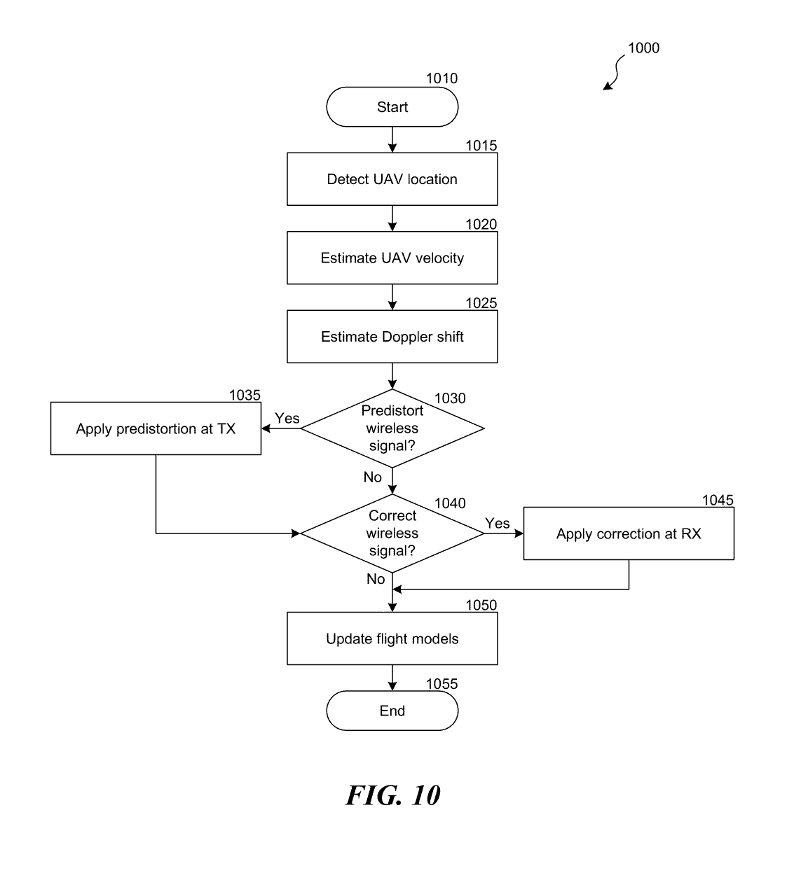

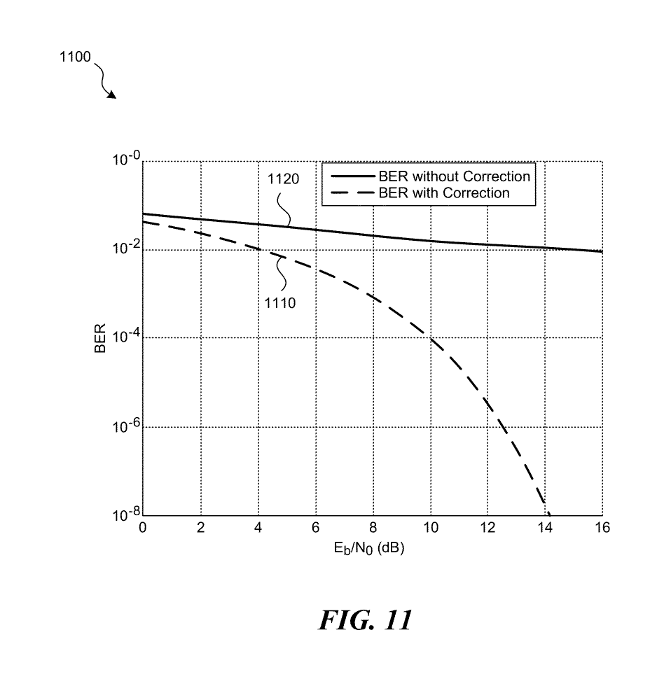

For the protein expression assay ITGA6 was selected based on qRT-PCR results and hapblock analysis. Additionally ITGB6 was chosen because it was significantly different in the hapblock analysis. Finally, CAPN1, CAPN2, and CAST were selected based on their established role in tenderness from the literature despite not being found significant in any previous analyses performed on these samples. Based on western blot assays, only the ITGA6 protein concentration was shown to be different between groups (P=0.045) (FIG. 4).

The tender group had a background corrected expression level 1.8 fold higher than the tough group (Table 18). This was roughly the same expression difference as was found among the mRNA. Protein expression levels for the other genes measured do not seem to closely match mRNA expression, possibly due to post transcriptional regulation or protein degradation.

TABLE 18
Background corrected mean fluorescence as determined by western blot
analysis

| | | | | Low/ | |
|---|---|---|---|---|---|
| | | High | Low | High | P-value |
| Protein | CAPN1 | 83,312 ± 7299 | 69,422 ± 3476 | 0.8 | 0.137 |
| Ex- | CAPN2 | 103,334 ± 2948 | 100,843 ± 4271 | 1.0 | 0.648 |
| pression | CAST | 116,740 ± 4489 | 118,315 ± 4229 | 1.0 | 0.807 |
| | ITGA6 | 81,237 ± 18413 | 149,840 ± 20002 | 1.8 | 0.045 |
| | ITGB1 | 94,742 ± 6956 | 85,622 ± 1210 | 0.9 | 0.244 |
| | ITGB6 | 44,066 ± 4276 | 50,586 ± 4174 | 1.2 | 0.317 |
| mRNA | CAPN1 | 1.0 ± 0.1 | 1.4 ± 0.2 | 1.5 | 0.052 |
| Ex- | CAPN2 | 0.9 ± 0.1 | 1.1 ± 0.1 | 1.2 | 0.111 |
| pression | CAST | 0.4 ± 0.1 | 0.5 ± 0.2 | 1.3 | 0.597 |
| | ITGA6 | 0.9 ± 0.1 | 1.6 ± 0.2 | 1.8 | 0.019 |
| | ITGB1 | 0.6 ± 0.4 | 0.9 ± 0.2 | 1.5 | 0.563 |
| | ITGB6 | 0.7 ± 0.4 | 1.7 ± 0.3 | 2.4 | 0.108 |

Values represent the mean protein fluorescence for the four samples selected for western blot analysis. Units given for protein fluorescence are optical density. Quantitative RT-PCR results are given for these four as a comparison.

Protein analysis of a subset of samples revealed that the ITGA6 protein was present at different levels between tenderness groups. Concentrations of ITGA6 protein in muscle samples of the more tender ES group were 1.8 fold greater than in the tougher ES group (P=0.045). Gene expression between these two groups was also 1.8 fold higher in the more tender group (P=0.019). ITGB6 protein expression was not significantly different between groups. Further, the CAPN1, CAPN2, and CAST proteins did not differ significantly in concentration between ES tenderness groups (P=0.137, 0.648, and 0.807, respectively). In the mRNA expression studies, the CAPN1, CAPN2, and CAST genes were also not found to vary significantly between ES tenderness groups (P=0.051, 0.937, and 0.379, respectively).

### Example 10

### Statistical Analysis

SPSS v16.0 was used for all statistical analysis with the exception of the microarray expression levels. Microarray analysis procedures were based on Genespring software. Normalization and quality control were performed using embedded functions in the Genespring GX v11.0.2 software. Quantile normalization was used to reduce variability between arrays. Array data were subjected to baseline normalization to the median expression level and had a minimum threshold of 1.0 expression all using standard quality control options built in to the Genespring software. Quality control on arrays was performed via correlation plots.

Unless otherwise noted an independent samples t-test was used to determine significance. Measures of averages are given as mean values. Estimations of error are given as standard error of the mean (SEM).

What is claimed is:

1. A method of selecting a head of beef cattle with a genetic predisposition for increased meat tenderness comprising selecting said head of beef cattle based on the presence in the genome of at least one genetic haplotype conferring said increased meat tenderness selected from the group consisting of
(a) a haplotype located in a region on bovine chromosome 2 defined by positions −24230649 and −24118992;
(b) a haplotype located in a region on bovine chromosome 2 defined by positions 103881402 and 103950562; and
(c) a haplotype located in a region on bovine chromosome 2 defined by positions 36242459 and 36362623.

2. The method of claim 1, comprising selecting said head of beef cattle based on the presence in the genome of at least two of said haplotypes.

3. The method of claim 1, wherein said haplotype of (a) is inherited from the maternal parent.

4. The method of claim 1, wherein said haplotype of (a) is inherited from the paternal parent.

5. The method of claim 1, wherein said haplotype of (a) is inherited from the genome of a Nellore head of cattle.

6. The method of claim 1, wherein said haplotype of (b) is inherited from the paternal parent.

7. The method of claim 1, wherein said haplotype of (b) is inherited from the genome of an Angus head of cattle.

8. The method of claim 1, wherein said haplotype of (c) is inherited from the maternal parent.

9. The method of claim 1, wherein said haplotype of (c) is inherited from the paternal parent.

10. The method of claim 1, wherein said haplotype of (c) is inherited from the genome of a Nellore head of cattle.

11. The method of claim 1, further comprising sequencing said haplotype.

12. The method of claim 1, further comprising detecting at least one SNP within or genetically linked to said at least one genetic haplotype, wherein said SNP is set forth in Tables 12-14.

13. The method of claim 12, further comprising detecting all SNPs within or genetically linked to said at least one haplotype.

14. The method of claim 1, further comprising genotyping at least one parent of said head of beef cattle for the presence of said at least one genetic haplotype.

15. The method of claim 1, comprising genotyping both parents of said head of beef cattle.

16. The method of claim 1, wherein said head of beef cattle is a Bos indicus or a Bos taurus head of beef cattle.

17. The method of claim 1, wherein said head of beef cattle is a hybrid of a Bos indicus species and a Bos taurus species.

18. The method of claim 17, wherein said Bos indicus species further comprises a Nellore head of cattle.

19. The method of claim 17, wherein said Bos taurus species further comprises an Angus head of cattle.

20. The method of claim 1, further comprising genotyping a population of beef cattle for the presence of said at least one genetic haplotype.

21. The method of claim 1, further comprising breeding the selected head of beef cattle comprising said at least one genetic haplotype with a second head of beef cattle to obtain a progeny head of beef cattle with desired meat tenderness relative to a head of beef cattle of the same breed lacking said at least one genetic haplotype.

22. The method of claim 21, further comprising breeding the progeny head of beef cattle with desired meat tenderness to a second head of beef cattle to produce a progeny of a further generation comprising said desired meat tenderness.

23. The method of claim 1, wherein the presence of said at least one genetic haplotype conferring said increased meat tenderness is detected by assaying of genetic material from the head of beef cattle.

24. The method of claim 1, wherein said haplotype of (a) comprises the ITGA6 gene.

25. The method of claim 1, wherein said haplotype of (b) comprises the FN1 gene.

26. The method of claim 1, wherein said haplotype of (c) comprises the ITGB6 gene.

27. The method of claim 23, wherein said assaying is carried out by PCR.

28. A method of predicting the tenderness of meat in a head of beef cattle after electrical stimulation comprising genotyping the head of beef cattle for the presence in the genome of at least one genetic haplotype conferring said increased meat tenderness selected from the group consisting of
(a) a haplotype located in a region on bovine chromosome 2 defined by positions −24230649 and −24118992;
(b) a haplotype located in a region on bovine chromosome 2 defined by positions 103881402 and 103950562; and
(c) a haplotype located in a region on bovine chromosome 2 defined by positions 36242459 and 36362623.

29. The method of claim 28, wherein said head of beef cattle is a Bos indicus or a Bos taurus head of beef cattle.

30. The method of claim 28, wherein said head of beef cattle is a hybrid between a Bos indicus species and a Bos taurus species.

31. The method of claim 30, wherein said Bos indicus species further comprises a Nellore head of cattle.

32. The method of claim 30, wherein said Bos taurus species further comprises further comprises an Angus head of cattle.

33. A method of determining the breeding value of a head of cattle comprising:
(a) genotyping the head of beef cattle to determine the presence in the genome of at least one genetic haplotype conferring said increased meat tenderness selected from the group consisting of
(i) a haplotype located in a region on bovine chromosome 2 defined by positions −24230649 and −24118992;
(ii) a haplotype located in a region on bovine chromosome 2 defined by positions 103881402 and 103950562; and
(iii) a haplotype located in a region on bovine chromosome 2 defined by positions 36242459 and 36362623;
(b) determining an estimated breeding value for the individual; and
(c) selecting at least a first individual with a desired estimated breeding value for breeding.

34. The method of claim 27, further comprising:
(d) breeding said individual to a second individual to obtain progeny.

35. A kit for identifying a head of beef cattle with increased meat tenderness following postmortem electrical stimulation, the kit comprising:
(a) at least one primer that amplifies a SNP within or genetically linked to at least one haplotype conferring said increased meat tenderness selected from the group consisting of
(i) a haplotype located in a region on bovine chromosome 2 defined by positions −24230649 and −24118992;
(ii) a haplotype located in a region on bovine chromosome 2 defined by positions 103881402 and 103950562; and
(ii) a haplotype located in a region on bovine chromosome 2 defined by positions 36242459 and 36362623;
wherein said SNP is set forth in Tables 12-14; and
(b) a reaction reagent.


SEQUENCE LISTING (78 sequences)

SEQ ID NO: 1 - DNA, 18 bp, Unknown
  Primer
tgccggagtctcgttcgt

SEQ ID NO: 2 - DNA, 20 bp, Unknown
  Primer
ggtgcatggccgttcttagt

SEQ ID NO: 3 - DNA, 17 bp, Unknown
  Primer
cgacggtcaggtcatca

SEQ ID NO: 4 - DNA, 21 bp, Unknown
  Primer
tgctgttgtaggtggtctcat

SEQ ID NO: 5 - DNA, 23 bp, Unknown
  Primer
aagatggaggagattggaaggat

SEQ ID NO: 6 - DNA, 22 bp, Unknown
  Primer
ggcttgtagttgacgatgctct

SEQ ID NO: 7 - DNA, 18 bp, Unknown
  Primer
ggtctttgacaacaagca

SEQ ID NO: 8 - DNA, 17 bp, Unknown
  Primer
tgatggttctggcgata

SEQ ID NO: 9 - DNA, 21 bp, Unknown
  Primer
cgggagacaagaactacatca

SEQ ID NO: 10 - DNA, 19 bp, Unknown
  Primer
cgtagagggcactggagaa

SEQ ID NO: 11 - DNA, 22 bp, Unknown
  Primer
agcagagcaaacaacagtccaa

SEQ ID NO: 12 - DNA, 19 bp, Unknown
  Primer
cctccagcgtcccattcat

SEQ ID NO: 13 - DNA, 19 bp, Unknown
  Primer
agtaagccgcagtggaggt

SEQ ID NO: 14 - DNA, 20 bp, Unknown
  Primer
cgcaaaacaccctgaagact

SEQ ID NO: 15 - DNA, 17 bp, Unknown
  Primer
gccgccttcagttggaa

SEQ ID NO: 16 - DNA, 23 bp, Unknown
  Primer
ggtctcccttgaggtcgtaagtg

SEQ ID NO: 17 - DNA, 20 bp, Unknown
  Primer
gaccataggcttcgctgtct

SEQ ID NO: 18 - DNA, 20 bp, Unknown
  Primer
aggttgatgaactgctcgga

SEQ ID NO: 19 - DNA, 22 bp, Unknown
  Primer
cgactggagacactgttcagga

SEQ ID NO: 20 - DNA, 24 bp, Unknown
  Primer
cttcaggcagattggttatcactt

SEQ ID NO: 21 - DNA, 21 bp, Unknown
  Primer
gctgtcgtctctgaagtggtt

SEQ ID NO: 22 - DNA, 23 bp, Unknown
  Primer
ggcatcgtcaagttctttgttgt

SEQ ID NO: 23 - DNA, 23 bp, Unknown
  Primer
gggcaacagcagattcacttaca

SEQ ID NO: 24 - DNA, 21 bp, Unknown
  Primer
tcaaggataggcaggcgagat

SEQ ID NO: 25 - DNA, 24 bp, Unknown
  Primer
tgaaaatgtgctcttgctgtgagt

SEQ ID NO: 26 - DNA, 24 bp, Unknown
  Primer
aatgaccttgacgatgaagtcctt

SEQ ID NO: 27 - DNA, 18 bp, Unknown
  Primer
tgcagccgccatttcttc

SEQ ID NO: 28 - DNA, 20 bp, Unknown
  Primer
tagcgctgttggacgctcta

SEQ ID NO: 29 - DNA, 18 bp, Unknown
  Primer
gctggagacggaggatga

SEQ ID NO: 30 - DNA, 24 bp, Unknown
  Primer
cactgagtcgttgatgtagacctt

SEQ ID NO: 31 - DNA, 20 bp, Unknown
  Primer
ctgaccaagactgccaccat

SEQ ID NO: 32 - DNA, 20 bp, Unknown
  Primer
atgttgttgcctgctttgct

SEQ ID NO: 33 - DNA, 19 bp, Unknown
  Primer
cgtctccaccaagttcgct

SEQ ID NO: 34 - DNA, 20 bp, Unknown
  Primer
tcacagatgctgccaacagt

SEQ ID NO: 35 - DNA, 22 bp, Unknown
  Primer
aagcaggcaccaatatgatgat

SEQ ID NO: 36 - DNA, 23 bp, Unknown
  Primer
ccacttgacgatgaggatgtagt

SEQ ID NO: 37 - DNA, 27 bp, Unknown
  Primer
cttccctaataatgttgaaccagttac

SEQ ID NO: 38 - DNA, 24 bp, Unknown
  Primer
gaacacacatctttctgtcgtgaa

SEQ ID NO: 39 - DNA, 23 bp, Unknown
  Primer
gcaagaatctcaacaactcgcaa

SEQ ID NO: 40 - DNA, 21 bp, Unknown
  Primer
gccagtcgctcatcggaaata

SEQ ID NO: 41 - DNA, 22 bp, Unknown
  Primer
aagacagacagatgatggcaga

SEQ ID NO: 42 - DNA, 22 bp, Unknown
  Primer
ggcacttgatgttcacacagtt

SEQ ID NO: 43 - DNA, 19 bp, Unknown
  Primer
agcctgtgaactgcctcaa

SEQ ID NO: 44 - DNA, 22 bp, Unknown
  Primer
acatcagccgtcagaacataat

SEQ ID NO: 45 - DNA, 22 bp, Unknown
  Primer
cttctattgctcaccttgtcca

SEQ ID NO: 46 - DNA, 24 bp, Unknown
  Primer
agataatgttcctaccgctgactt

SEQ ID NO: 47 - DNA, 20 bp, Unknown
  Primer
cacaacctccagcagaccaa

SEQ ID NO: 48 - DNA, 21 bp, Unknown
  Primer
ccatcagcacagtgtccacaa

SEQ ID NO: 49 - DNA, 20 bp, Unknown
  Primer
cggagagagttcgccaagtt

SEQ ID NO: 50 - DNA, 24 bp, Unknown
  Primer
ctgtgccattgtaggatttgttga

SEQ ID NO: 51 - DNA, 23 bp, Unknown
  Primer
catcaatgagaaagactgtccaa

SEQ ID NO: 52 - DNA, 23 bp, Unknown
  Primer
caataagaagaatagccagcgaa

SEQ ID NO: 53 - DNA, 17 bp, Unknown
  Primer
atacgcacagccgcaga

SEQ ID NO: 54 - DNA, 21 bp, Unknown
  Primer
cacgatgtcacccacctctaa

SEQ ID NO: 55 - DNA, 20 bp, Unknown
  Primer
tgaggaagcggaggaacaat

SEQ ID NO: 56 - DNA, 18 bp, Unknown
  Primer
tgggactcggcaatgtca

SEQ ID NO: 57 - DNA, 20 bp, Unknown
  Primer
caatgacctgacaacccaga

SEQ ID NO: 58 - DNA, 22 bp, Unknown
  Primer
ccttgacaactgagacaccaga

SEQ ID NO: 59 - DNA, 25 bp, Unknown
  Primer
gctaatccagaaccactttgtagat

SEQ ID NO: 60 - DNA, 18 bp, Unknown
  Primer
gccttcgcctgtcctcat

SEQ ID NO: 61 - DNA, 22 bp, Unknown
  Primer
aagttgtggcagtgtcagtgga

SEQ ID NO: 62 - DNA, 23 bp, Unknown
  Primer
cgtagtctcgggaaatctgtttg

SEQ ID NO: 63 - DNA, 20 bp, Unknown
  Primer
accccaatgagaccaatgaa

SEQ ID NO: 64 - DNA, 23 bp, Unknown
  Primer
gcagtaccctttcgcttacctat

SEQ ID NO: 65 - DNA, 25 bp, Unknown
  Primer
gactggagatgaatacaatgtggaa

SEQ ID NO: 66 - DNA, 23 bp, Unknown
  Primer
cgtttggtactgtgagggataga

SEQ ID NO: 67 - DNA, 17 bp, Unknown
  Primer
accagccgcaacgtgaa

SEQ ID NO: 68 - DNA, 17 bp, Unknown
  Primer
accagccgcaacgtgaa

SEQ ID NO: 69 - DNA, 20 bp, Unknown
  Primer
gcagaacctgatgctttgtg

SEQ ID NO: 70 - DNA, 19 bp, Unknown
  Primer
cgtaggagagcgtgtgctt

SEQ ID NO: 71 - DNA, 21 bp, Unknown
  Primer
aaatgccaagaacggaaactt

SEQ ID NO: 72 - DNA, 21 bp, Unknown
  Primer
gtcagacacgccaaccagata

SEQ ID NO: 73 - DNA, 21 bp, Unknown
  Primer
gctgggacataaggtgacaca

SEQ ID NO: 74 - DNA, 18 bp, Unknown
  Primer
ctgcgagcgtcttggtct

SEQ ID NO: 75 - DNA, 26 bp, Unknown
  Primer
aaatagccttcagggagaaagtgtaa

SEQ ID NO: 76 - DNA, 26 bp, Unknown
  Primer
aatgcttccttaaatgttcggtcttc

SEQ ID NO: 77 - DNA, 20 bp, Unknown
  Primer
gcatcccacagactatttcc

SEQ ID NO: 78 - DNA, 20 bp, Unknown
  Primer
gcaaagacaatgacagacca